SEQUENCE LISTING (32 sequences)

SEQ ID NO: 1 - DNA, 33 bp, Artificial
  Probe APC 1-WT for the APC gene from the human genome.
aaaaaaaaaaaaaaaaaaaagcagaaataaaag

SEQ ID NO: 2 - DNA, 33 bp, Artificial
  Probe APC 1-MUT for the APC gene from the human genome.
aaaaaaaaaaaaaaaaaaaagcagaaaaaaaag

SEQ ID NO: 3 - DNA, 35 bp, Artificial
  Probe APC 2 for the APC gene from the human genome.
aaaaaaaaaaaaaaaaaaaaaaaagattggaacta

SEQ ID NO: 4 - DNA, 31 bp, Artificial
  Probe Factor V 1-WT for the APC gene from the human genome.
aaaaaaaaaaaaaaaaaaaatattcctcgcc

SEQ ID NO: 5 - DNA, 30 bp, Artificial
  Probe Factor V 1-MUT for the APC gene from the human genome.
aaaaaaaaaaaaaaaaaaaaattccttgcc

SEQ ID NO: 6 - DNA, 40 bp, Artificial
  Probe Factor V 2 for the APC gene from the human genome.
aaaaaaaaaaaaaaaaaaaactgctcttacagattagaag

SEQ ID NO: 7 - DNA, 119 bp, Artificial
  MTHFR gene 119 PCR amplicon
tattggcaggttaccccaaaggccaccccgaagcagggagctttgaggctgacctgaagcacttgaaggagaaggtgtctgcgggagccgatttcatcatcacgcagcttttctttgag

SEQ ID NO: 8 - DNA, 78 bp, Artificial
  APC gene 78 base sequence - Wild type  (1)
cgctcacaggatcttcagctgacctagttccaatcttttcttttatttctgctatttgcagggtattagcagaatctg

SEQ ID NO: 9 - DNA, 78 bp, Artificial
  APC gene 78 base sequence - Mutant (2)
cgctcacaggatcttcagctgacctagttccaatcttttcttttttttctgctatttgcagggtattagcagaatctg

SEQ ID NO: 10 - DNA, 52 bp, Artificial
  Factor V Leiden 99 bp PCR product
gacatcgcctctgggctaataggactacttctaatctgtaagagcagatccc

SEQ ID NO: 11 - DNA, 21 bp, Artificial
  Probe MecA 1 for the mecA gene.
tggcatgagtaacgaagaata

SEQ ID NO: 12 - DNA, 21 bp, Artificial
  Probe MecA 2 for the mecA gene.
ttccagattacaacttcacca

SEQ ID NO: 13 - DNA, 86 bp, Artificial
  Target MecA 3 target from the mecA gene.
tggtgaagttgtaatctggaacttgttgagcagaggttcttttttatcttgggttaatttattatattcttcgttactcatgccat

SEQ ID NO: 14 - DNA, 281 bp, Artificial
  Target MecA 4 is a mecA 281 base-pair PCR amplicon.
atccaccctcaaacaggtgaattattagcacttgtaagcacaccttcatatgacgtctatccatttatgtatggcatgagtaacgaagaatataataaattaaccgaagataaaaaagaacctctgctcaacaagttccagattacaacttcaccaggttcaactcaaaaaatattaacagcaatgattgggttaaataacaaaacattagacgataaaacaagttataaaatcgatggtaaaggttggcaaaaagataaatcttggggtggttacaacgt

SEQ ID NO: 15 - DNA, 21 bp, Artificial
  Probe mecA 3 for the mecA gene.
aaagaacctctgctcaacaag

SEQ ID NO: 16 - DNA, 22 bp, Artificial
  Probe mecA 4 for the mecA gene.
gcacttgtaagcacaccttcat

SEQ ID NO: 17 - DNA, 22 bp, Artificial
  Probe 1 for the mecA gene.
atggcatgagtaacgaagaata

SEQ ID NO: 18 - DNA, 20 bp, Artificial
  Probe 2 for the mecA gene.
ttccagattacacttcacca

SEQ ID NO: 19 - DNA, 85 bp, Artificial
  DNA target.
tggtaagttgtaatctggaacttgttgagcagaggttcttttttatcttcggttaatttattatattcttcgttactcatgccat

SEQ ID NO: 20 - DNA, 30 bp, Artificial
  Probe 1 for the human coagulation Factor V gene.
aaaaaaaaaatggacaggcgaggaatacag

SEQ ID NO: 21 - DNA, 35 bp, Artificial
  Probe 2 for the human coagulation Factor V gene.
tgatgcccagtgcttaacaagaccatactacagtg

SEQ ID NO: 22 - DNA, 250 bp, Artificial
  DNA Target
cttataagtggaacatcttagagtttgatgaacccacagaaaatgatgcccagtgcttaacaagaccatactacagtgacgtggacatcatgagagacatcgcctctgggctaataggactacttctaatctgtaagagcagatccctggacaggcgaggaatacaggtattttgtccttgaagtaacctttcagaaattctgagaatttcttctggctagaacatgttaggtctcctggctaaataatg

SEQ ID NO: 23 - DNA, 20 bp, Artificial
  T20 oligonucleotide
tttttttttttttttttttt

SEQ ID NO: 24 - DNA, 10 bp, Artificial
  A10-aptamer
aaaaaaaaaa

SEQ ID NO: 25 - DNA, 54 bp, Artificial
  An anti-IgE aptamer
aaaaaaaaaacgcggggcacgtttatccgtccctcctagtggcgtgccccgcgc

SEQ ID NO: 26 - DNA, 20 bp, Artificial
  T20 diluent
tttttttttttttttttttt

SEQ ID NO: 27 - DNA, 40 bp, Artificial
  Oligo 1
ttccagattacacttcaccatttttttttttttttttttt

SEQ ID NO: 28 - DNA, 41 bp, Artificial
  Oligo 2
aaagaacctctgctcaacaagtttttttttttttttttttt

SEQ ID NO: 29 - DNA, 30 bp, Artificial
  Probe 1 for the mecA gene
aaaaaaaaaaaaaaaaaaaaaaaaaaaaaa

SEQ ID NO: 30 - DNA, 86 bp, Artificial
  DNA target
tggtgaagttgtaatctggaacttgttgagcagaggttcttttttatcttcggttaatttattatattcttcgttactcatgccat

SEQ ID NO: 31 - DNA, 20 bp, Artificial
  Deoxyadenosine Spacer
aaaaaaaaaaaaaaaaaaaa

SEQ ID NO: 32 - DNA, 15 bp, Artificial
  Deoxyadenosine Spacer
aaaaaaaaaaaaaaa

(19) United States
(12) Patent Application Publication
(10) Pub. No.: US 2005/0250094 A1
(43) Pub. Date: Nov. 10, 2005

(54) Method for detecting analytes based on evanescent illumination and scatter-based detection of nanoparticle probe complexes

(75) Inventors: James J. Storhoff, Evanston, IL (US); Adam Lucas, Arlington Heights, IL (US); Uwe R. Muller, Waukegan, IL (US); Yijia Paul Bao, Mount Prospect, IL (US); Michael Senical, Wheeling, IL (US); Viswanadham Garimella, Vernon Hills, IL (US)

Correspondence Address: MCDONNELL BOEHNEN HULBERT & BERGHOFF LLP, 300 S. WACKER DRIVE, 32ND FLOOR, CHICAGO, IL, 60606, US

(73) Assignee: Nanosphere, Inc.

(21) Appl. No.: 10/995,051

(22) Filed: Nov. 22, 2004

Related U.S. Application Data
(63) Continuation-in-part of application No. 10/854,848, filed on May 27, 2004.
(60) Provisional application No. 60/474,569, filed on May 30, 2003.
(60) Provisional application No. 60/499,034, filed on Aug. 29, 2003.
(60) Provisional application No. 60/517,450, filed on Nov. 4, 2003.
(60) Provisional application No. 60/567,874, filed on May 3, 2004.

(51) Int. Cl. C12Q 1/70; C12Q 1/68; G01N 33/551

(52) U.S. Cl. 435/5; 435/6; 436/524

(57) ABSTRACT

The invention provides methods of detecting one or more specific binding analytes, such as nucleic acids and proteins, in the presence of a neutral or anionic polysaccharide, through light scattering techniques, where a change in light scattering caused by the formation of nanoparticle label complexes within the penetration depth of the evanescent wave of a wave guide signals the presence of the analyte.

### CROSS-REFERENCE

This application is a Continuation-in-Part Application of U.S. application Ser. No. 10/854,848 filed May 27, 2004, which claims the benefit of priority from U.S. Provisional application Nos. 60/474,569 filed May 30, 2003, 60/499,034, filed Aug. 29, 2003, and 60/517,450 filed Nov. 4, 2003, all of which are incorporated by reference in their entirety. This application also claims the benefit of priority from U.S. Provisional application (60/567,874, filed May 3, 2004), which is incorporated by reference in its entirety.

The work described in this application was supported in part by the National Institutes of Health, National Cancer Institute, under Grant No. 2 R44 CA85008-02. Accordingly, the United States Government may have certain rights to the invention described and claimed herein.

### FIELD OF THE INVENTION

The present invention relates to specific binding partner interactions, evanescent waveguides and light scattering. More particularly, the present invention relates to a method for detecting one or more specific binding analytes, e.g., nucleic acids or proteins, in the presence of a neutral or anionic polysaccharide, through light scattering techniques, where a change in light scattering caused by the formation of nanoparticle label complexes, within the penetration depth of the evanescent wave of a wave guide, signals the presence of an analyte.

### BACKGROUND OF THE INVENTION

Nucleic acid based analysis has become an increasingly important tool for the diagnosis of genetic and infectious diseases¹⁻³. Assays that utilize target amplification procedures, such as polymerase chain reaction (PCR), in conjunction with fluorescently labeled probes have gained widespread acceptance as the detection method of choice^{4,5}, but have the drawback of relatively complex and expensive assay and instrumentation configurations. A number of novel labeling and detection methodologies have been developed to circumvent these limitations. For example, signal amplification through enzymatic cleavage of fluorophore labeled probes has enabled detection of specific DNA sequences at sub-attomole levels within complex mixtures⁶. Alternatively, a calorimetric response generated by enzyme catalysis on optically coated silicon substrates has been utilized for high sensitivity nucleic acid detection without instrumentation⁷. Nonetheless, these methods still require complex enzyme-based signal amplification procedures and/or fluorescence readers to achieve sufficient sensitivity. For wide-spread adoption into clinical diagnostics and especially screening procedures there is a clear need for simpler nucleic acid detection that does not rely on enzymatic target or signal amplification to provide for sufficiently high sensitivity and specificity. Furthermore, detection strategies that provide high sensitivity and specific detection of other bioanalytes such as proteins would offer similar advantages in diagnostic applications.

Mirkin and coworkers previously reported a new calorimetric detection method for nucleic acids based on the distance dependent optical properties of DNA-modified gold nanoparticles (DNA-GNP)⁸⁻¹⁰. Making use of the fact that the absorption frequency of the surface plasmon band of metal nanoparticles is dependent on interparticle distance as well as aggregate size, a DNA hybridization mediated aggregation of nanoparticles was shown to result in a red-shift of their surface plasmon band and a visual change of solution color from red to purple or blue. This visible color change could be observed and permanently recorded by spotting a small aliquot (e.g. 1 μL) of the hybridization solution onto a reverse phase TLC plate⁹. More recently this strategy has been extended to the detection of proteins¹¹, carbohydrates¹², and metal ions^{13,14} using suitably functionalized gold nanoparticles. Because DNA-GNP probes have unique hybridization characteristics, namely sharp melting transitions and raised Tₘ's, this detection method achieved a remarkable sequence specificity that allowed discrimination of single base mismatches, deletions, or insertions¹⁵.

Though the simplicity of spotting the sample followed by visual readout is extremely attractive for diagnostic applications, the relatively low limit of detection (LOD) of 10 fmol target⁹ has limited its utility to date. Two factors that contribute to the low sensitivity are the inability to detect nanoparticles at lower concentrations, and the requirement of a larger aggregate to achieve a detectable calorimetric shift. Experimental data and optical modeling of the DNA-linked gold nanoparticle structures has demonstrated that a large number (e.g. hundreds to thousands) of 15 nm diameter gold particles are needed to provide a measurable red-shift in the surface plasmon band^{10,16}. Experimentally, this necessitates a molar excess of target over nanoparticles to promote formation of large aggregates¹⁰. Efforts to increase sensitivity have focused on using 50-100 nm diameter gold particles which absorb more light than the 15 nm diameter particles. However, pre- and post-test (aggregation) colors were not easily distinguishable by a visual readout after spotting¹⁷. Furthermore, using hybridization conditions well known in the art for nucleic acid detection, gold nanoparticle probe complexes are not formed with more complex nucleic acid samples such as PCR amplicons, which are double stranded. Snap freezing the sample accelerates the formation of probe-target complexes with short PCR amplicons (<150 base-pairs). However, freezing is not amenable to automation and promotes mismatch formation at the lower temperature, and therefore, it is not suitable for more complex target analyte samples such as PCR amplicons with high GC content, protein-based reactions where continuous freeze-thaw reactions may damage the protein or antibody, or for genomic DNA samples where a large number of non-target sequences are present. There is a need in the art to develop methods for accelerating the formation of nanoparticle-probe complexes that does not require freezing. In addition, there is a need in the art for the development of more sensitive tests based on nanoparticle probe complexes that can utilize unamplified genomic DNA samples or low concentrations of other biological analytes (e.g proteins, cells, or chromosomes) with detection in a homogeneous format.

The detection of protein analytes has emerged as a powerful tool for proteomics as well as diagnostics.¹⁸⁻²⁰ A variety of different detection methods have been developed for labeling antibody arrays including, but not limited to, fluorescence,^{18,21} chemiluminescence,¹⁹ resonance light scattering,²⁰ and SERS.²² Signal amplification strategies such as rolling circle amplification (RCA) also have been used to increase the detection sensitivity of fluorescence-based strategies.^{23,24} These methods have provided high sensitivity detection (<10 pg/mL) of protein analytes, but the use of such labeling strategies has been limited by the performance of the antibodies which are prone to cross reactivity.²⁰ In addition, the reproducible preparation of highly purified antibody reagents is both challenging and time consuming.²⁵ It would be extremely beneficial to develop a probe system that provides not only high sensitivity and specificity for the protein analyte of interest, but also reproducibility in production and use.

DNA can be conjugated to gold nanoparticles via a thiol linkage⁸ and the resulting DNA modified gold particles can be used to detect DNA targets, as well as other analytes, in a variety of formats,^{14,26,27} including DNA microarrays, where high detection sensitivity is achieved in conjunction with silver amplification.^{28,29} Additional key features of this technology include the remarkable stability and robustness of the DNA-modified gold nanoparticles which withstand both elevated temperatures and salt concentrations,^{8,30} as well as the remarkable specificity by which DNA sequences are recognized.^{15,31} Although prior studies have demonstrated that antibodies or haptens can be attached to gold nanoparticles through DNA-directed immobilization or passive adsorption and used for protein detection,^{20,32,33} these strategies are still prone to the limitations cited above. It would be a significant advance to use the DNA-modified gold particles directly for protein analyte detection. RNA and DNA aptamers can substitute for monoclonal antibodies in various applications (Jayasena, “Aptamers: an emerging class of molecules that rival antibodies in diagnostics.” Clin. Chem., 45(9):1628-50, 1999; Morris et al., “High affinity ligands from in vitro selection: complex targets.” Proc. Natl. Acad. Sci., USA, 95(6):2902-7, 1998). Aptamers are nucleic acid molecules having specific binding affinity to non-nucleic acid or nucleic acid molecules through interactions other than classic Watson-Crick base pairing. Aptamers are described, for example, in U.S. Pat. Nos. 5,475,096; 5,270,163; 5,589,332; 5,589,332; and 5,741,679.

During the past 15 years, fluorescent in situ hybridization (FISH) has emerged as one of the most important cytogenetic tools for the analysis of genetic aberrations (Iqbal et al., 1999, East Mediterr Health J 5:1218-24). Since it provides a 100 fold increase in resolution over standard karyotyping, it has now become the standard of care in prenatal diagnostics as well as the molecular analysis of many cancers (see for example, King et al., 2000, Mol Diagn 5:309-19 and Bartlett et. al, 2003, J Pathol 199: 411-7). FISH consists in the hybridization of fluorescently labeled DNA probes (labeled either directly or indirectly) to specific chromosomal targets that can be in form of condensed metaphase chromosomes or in much less condensed interphase DNA. Detection is via fluorescent microscopy. FISH probes are classified into 3 types of probes, based on the size of the target sequence. Painting probes or Whole Chromosome Paints (WCP's) consist in a pool of many different sequences which together decorate a whole chromosome from one end to the other. CEP's or Centromere Enumeration Probes are typically composed of simple repeat sequences that are specific to the centromere of a given chromosome (alpha satellite probes). Location specific identifier (LSI) probes typically span a target size of ˜100 Kb and are designed to detect the copy number of specific genes.

There are several severe drawbacks and limitations to FISH that can be overcome by generating DNA probes with nanoparticles, as decribed below. The most significant handicap of the current FISH technology is sensitivity, which results in limited sequence resolution. For example, the Vysis LSI SRY DNA FISH probe consist of a 120 kb LSI targeting the 600 b SRY gene. The reason that a 120 kb sequence is needed to light up a 600 b region is sensitivity, in other words, the number of fluorochromes that can be attached per probe is limited and the number of probes that can be successfully hybridized to a chromosomal region is limited as well. The result is that for typical fluorescent microscopes, filter set combinations and fluorochrome lifetimes a target sequence of at least 50-100 kb is needed to generate sufficient signal above background in the average laboratory. This means that sequences changes smaller than ˜20 kb can not be detected. For M-FISH or SKY the resolution limit is even on the order of 1,000-2,000 kb (Saracoglu et al., 2001, Cytometry 44:7-15). Only in special situations where the chromosomal target sequence is highly repeated or where it has been stretched and stripped of all proteins (fiber FISH) can a resolution of 1-2 kb be reached (de Jong et. al., 1999, Trends Plant Sci., 4, 258-263 and Palotie et. al, 1996, Ann Med., 28, 101-106.). Consequently, there is a need in the art for more sensitive detection methods.

### SUMMARY OF THE INVENTION

The present invention provides a method for detection of specific binding analytes based on analyte mediated formation of metallic nanoparticle-labeled probe complexes, e.g., gold nanoparticle probe complexes, that results in a change in the color and/or intensity of light scattered, which can be measured by placing a small amount of the sample onto a waveguide and detecting the light scattered visually or with a photosensor. A schematic illustration of this detection method applied to nucleic acid detection is shown in FIG. 1. The nanoparticle probe complexes comprise two or more probes bound to a specific target analyte.

The invention also provides methods of homogeneous detection of target nucleic acid sequences in a sample without enzymatic target or signal amplification. In one aspect, the method comprises contacting a target nucleic acid sequence in the sample with at least one detector probe and observing a detectable change, wherein the sample comprises nucleic acid molecules of higher biological complexity relative to amplified nucleic acid molecules.

In one aspect of the invention, probe complexes comprise at least one type of nanoparticle probe bound to more than one region, e.g., a repeated region, of the specific target, e.g., a DNA molecule. In a particular aspect, the nanoparticle probes can have at least one type of specific binding member bound thereto, e.g., an oligonucleotide, that is complementary to a repeated region of a target, e.g., DNA. In another aspect, the nanoparticle probes can have two or more specific binding members bound thereto that are complementary to different portions of a target. In another aspect of the invention, the probe complexes comprise at least two types of nanoparticle probes bound to more than one region, e.g., a repeated region, of the specific target, e.g., a DNA molecule. Each type of nanoparticle probes can have at least one type of specific binding member, e.g., an oligonucleotide, that binds to at least one region of the specific target, e.g., a DNA molecule.

The scatter-based calorimetric detection methods of the invention provide much higher sensitivity (>4 orders of magnitude) in nucleic acid detection than the previously reported absorbance-based spot test when coupled to an improved hybridization method based on neutral or anionic polysaccharides that enables probe-target binding at low target concentrations. Moreover, the methods of the invention enable the detection of probe-target complexes containing two or more particles in the presence of a significant excess of non-complexed particles, which drives hybridization in the presence of low target concentrations. Also, dextran sulfate mediated probe-target complex formation in conjunction with evanescent induced scatter as provided herein enables a simple homogeneous hybridization and calorimetric detection protocol for nucleic acid sequences in total bacterial DNA, or with antibody-antigen interactions.

The invention further provides methods of detecting for the presence or absence of a single target molecule comprising: (a) providing at least two nanoparticles having specific binding complements of a single target molecule attached thereto, the specific binding complements being capable of recognizing at least two different portions of the single target molecule; (b) forming a light scattering complex by contacting a sample believed to contain the specific binding complement with the nanoparticles under conditions effective to allow binding of the specific binding complements to two or more portions of the single target molecule; (c) illuminating the light scattering complex under conditions effective to produce scattered light from said complex; and (d) detecting the light scattered by said light scattering complex as a measure of the presence of the single target molecule.

In certain aspects, the single target molecule from the sample can be isolated and immobilized on a substrate prior to being contacted with the nanoparticles. In other aspects, the single target molecule and the specific binding complement can be complements of a specific binding pair. The complements of a specific binding pair can comprise nucleic acid, oligonucleotide, peptide nucleic acid, polypeptide, antibody, antigen, carbohydrate, protein, peptide, amino acid, surface antigen, hormone, steroid, vitamin, drug, virus, polysaccharides, lipids, lipopolysaccharides, glycoproteins, lipoproteins, nucleoproteins, oligonucleotides, antibodies, immunoglobulins, albumin, hemoglobin, coagulation factors, peptide and protein hormones, non-peptide hormones, interleukins, interferons, cytokines, peptides comprising a tumor-specific epitope, cells, cell-surface molecules, microorganisms, fragments, portions, components or products of microorganisms, small organic molecules, nucleic acids and oligonucleotides, metabolites of or antibodies to any of the above substances. The nucleic acid and oligonucleotide can comprise genes, viral RNA and DNA, bacterial DNA, fungal DNA, mammalian DNA, cDNA, mRNA, RNA and DNA fragments, oligonucleotides, synthetic oligonucleotides, modified oligonucleotides, single-stranded and double-stranded nucleic acids, natural and synthetic nucleic acids, and aptamers.

In certain aspects, the single target molecule can be a nucleic acid and the specific binding complement can be an oligonucleotide. In still other aspects, the single target molecule can be a protein or antibody and the specific binding complement can be an antibody. In further aspects, the single target molecule is a gene sequence from a chromosome and the specific binding complements are oligonucleotides, the oligonucleotides having a sequence that is complementary to at least a portion of the gene sequence.

The invention also provides methods of detecting for the presence or absence of a target analyte having at least two portions comprising: (a) providing a type of nanoparticle having a specific binding complement of a target analyte attached thereto, the specific binding complement being capable of recognizing at least two different segments of the target analyte; (b) forming a light scattering complex by contacting a sample believed to contain the specific binding complement with the nanoparticle and with (i) a reagent that excludes volume or (ii) a reagent that accelerates DNA renaturation under conditions effective to allow binding of the specific binding complement to two or more portions of the target analyte; (c) illuminating the light scattering complex under conditions effective to produce scattered light from said complex; and (d) detecting the light scattered by said complex as a measure of the presence of the target analyte. In certain aspects, the reagent that excludes volume or that accelerates DNA renaturation is a polymer. In other aspects, the polymer is a neutral or polyanionic polysaccharide. In still other aspects, the neutral or polyanionic polysaccharide is a dextran sulfate polymer.

The invention also provides intermediate oligonucleotides that comprise a first portion complementary to the target analyte, and a second portion complementary to a binding complement of a nanoparticle, wherein the intermediate oligonucleotide can bind to the target analyte and the nanoparticle binding complement sequentially or simultaneously. These intermediate oligonucleotides or “universal nucleic acid tags” can be used to aid the detection of binding two or more metal nanoparticle probes to a target biomolecule. Two major advantages of this detection methodology when compared to a direct target binding system are: 1) a single nanoparticle probe can be used for detection by binding multiple intermediates to each target, and 2) multiple targets can be detected using a single gold probe via different target specific intermediates. In some aspects, an intermediate probe can comprise protein that can bind to both a target analyte and to a nanoparticle based probe of the invention.

Specific preferred embodiments of the invention will become evident from the following more detailed description of certain preferred embodiments and the claims.

### DESCRIPTION OF THE FIGURES

FIG. 1: Detection of gold probe complexes by evanescent illumination and scatter detection in the presence of neutral or anionic polysaccharides.

FIG. 2: Detection of a Factor V Leiden gene sequence using 40 nm gold particle probes (SEQ ID NO: 4 and 6) and scatter-based detection using different concentrations of dextran sulfate. A PCR-amplified 99 base-pair fragment (SEQ ID NO: 10) was used as a positive hybridization control, and an MTHFR 119 base-pair fragment (SEQ ID NO: 7) was used as a negative control. The dextran sulfate concentration was varied from 0-5% for detection.

FIG. 3: Detection of a mecA gene PCR product (1.3×10 copies/μL) with 40 nm diameter gold probes (SEQ ID NO: 11 and 12) at various concentrations of dextran sulfate. A no target control was used for comparison. Color CMOS image of a 1 μL sample aliquot spotted onto a glass slide is shown.

FIG. 4: I1307K genotyping using 30 nm gold particle probes. PCR-amplified 78 base-pair fragments of wild-type (wt), mutant (mut), or heterozygous (het) genotypes were tested with wild-type (WT) and mutant (MUT) APC gene probes. (A) Images taken with color CMOS after white light illumination. Og=orange. (B) Images taken with black and white CMOS after white light illumination (NIS 2000a). (C) Images taken with black and white CMOS after illumination with a red light emitting diode (LED).

FIG. 5: I1307K genotyping using 40 nm gold particle probes. PCR-amplified 78 base-pair fragments of wild-type (wt), mutant (mut), or heterozygous (het) genotypes were tested with wild-type (WT) and mutant (MUT) APC gene probes. (A) Images taken with color CMOS after white light illumination. Og=orange, Gr=green. (B) Images taken with black and white CMOS after white light illumination (NIS 2000a). (C) Images taken with black and white CMOS after illumination with red LED.

FIG. 6: I1307K genotyping using 50 nm gold particle probes. PCR-amplified 78 base-pair fragments of wild-type (wt), mutant (mut), or heterozygous (het) genotypes were tested with wild-type (WT) and mutant (MUT) APC gene probes. (A) Images taken with color CMOS after white light illumination. Og=orange, Gr=green. (B) Images taken with black and white CMOS after white light illumination (NIS 2000a). (C) Images taken with black and white CMOS after illumination with red LED.

FIG. 7: Detection of a mecA gene sequence (SEQ ID NO: 13) with 50 nm gold probes (SEQ ID NO: 11 and 12). A 1 ul sample aliquot was dried onto the slide for imaging. Og=orange. Yw=yellow. Gr=green.

FIG. 8: Analysis of gold probe—target complexes using a diode array detector. A 1 ul. sample aliquot was dried onto a glass slide, and the evanescent induced scatter from each sample was analyzed using a diode array detector.

FIG. 9: A mecA gene sequence (SEQ ID NO: 13) challenged with a single complementary gold probe (SEQ ID NO: 11 or 12) or two complementary gold probes (SEQ ID NO: 11 and 12). A solution with both probes in the absence of target served as a negative hybridization control. Og=orange. Gr=green.

FIG. 10. Gene-specific detection of methicillin resistance (mecA) from unamplified genomic DNA samples of Methicillin-resistant Staph. aureus (MRSA). Methicillin-sensitive Staph. aureus (MSSA) was used as a negative control. A 1 uL sample aliquot was spotted on the glass slide for both visual and color CMOS imaging. Gr=green, Yw=Yellow.

FIG. 11. Gene-specific detection of methicillin resistance (mecA) from unamplified genomic DNA samples of Methicillin resistant Staph. aureus (MRSA) and Methicillin susceptible Staph. aureus (MSSA). A 1 uL aliquot was dried onto the slide for imaging. Gr=green, Og=Orange.

FIG. 12. Colorimetric analysis using optical instrumentation. Methicillin resistant S. aureus (MRSA), S. epidermidis (MRSE), and methicillin sensitive S. aureus (MSSA) genomic DNA samples were tested using 40 nm gold probes. (a) The net signal intensity from the red channel of the color CMOS image (inset) is plotted for each sample. Three replicates were performed for each sample. The error bar represents the standard deviation in signal intensity. In the captured image, colorimetric scatter from all spots in row 1 (MRSA) is orange, row 2 (MSSA) is green, and row 3 (MRSE) is orange. (b) The glass slide is excited with a red LED, and the image is captured with a monochrome photosensor. The inset shows the captured image in this detection configuration.

FIG. 13. Schematic illustrating detection of target analyte using four gold probes per target.

FIG. 14. Highly sensitive gene-specific detection of methicillin Resistance (mecA) from unamplified genomic DNA samples of Methicillin resistant Staph. aureus (MRSA) using four 50 nm diameter gold probes. Methicllin susceptible Staph. aureus (MSSA) served as a negative control. A 1 uL aliquot was dried on the slide for imaging. Gr=green, Og=Orange.

FIG. 15. Highly sensitive gene-specific detection of methicillin resistance (mecA) from unamplified genomic DNA samples using four 50 nm diameter gold probes. A 1 uL aliquot was dried on the slide for imaging. MRSA=Methicillin-Resistant Staph. aureus, MSSA=Methicillin-Sensitive Staph. aureus, MRSE=Methicillin-Resistant Staph. epidermidis, MSSE=Methicillin Susceptible Staph. epidermidis, Gr=green, Og=Orange, Yw=Yellow.

FIG. 16: Schematic of change in scatter color produced by binding of antibody coated gold particles to a protein target. IgE antibody target is shown as an example.

FIG. 17: Detection of IgE target using anti-IgE coated 50 nm gold probes in conjunction with dextran sulfate to promote probe-target complex formation. The scatter color from IgE target samples was compared to IgG control samples with and without dextran sulfate. The images were recorded with a color CMOS photosensor after evanescent illumination with white light. It should be noted that the scatter color from the spotted samples is also detectable visually with the naked eye. The observed scatter colors are denoted by letters where Og=orange, Gr=green, Yw=yellow.

FIG. 18: Evanescent illumination and scatter-based detection of IgE target using anti-IgE coated 40 nm gold probes. The scatter color from a serial dilution of target was compared to control samples containing either no target or IgG. The IgE target concentration is shown above the figure, and the target/probe ratio is shown below the image. The images were recorded with a color CMOS photosensor. It should be noted that the scatter color from the spots is also detectable visually with the naked eye. The observed scatter colors are denoted by letters where Og=orange, Gr=green, Yw-Gr=yellowish green.

FIG. 19: Detection of evanescent-induced scatter from individual probe complexes spotted on a glass waveguide using high resolution optics.

FIG. 20: Schematic of change in scatter color based on the binding of two or more DNA-modified gold nanoparticle probes to a nucleic acid target immobilized onto a waveguide surface.

FIG. 21: Binding of one or two DNA-modified gold nanoparticle probes to a surface immobilized nucleic acid target. A) Color CCD images of gold probes (probe 1, probe 2, or probes 1+2) bound to the target recorded with an optical microscope after evanescent excitation with white light. B) Images of the red channel portion of the color CCD images. C) The number of scattering entities observed in the red channel as a function of number of probes bound per target.

FIG. 22: Schematic illustrating the preparation of aptamer coated gold probe arrays on a glass surface. Step one: DNA is immobilized onto a glass surface. A T₂₀ oligonucleotide is used in this example. Step two: An A₁₀-anti-IgE aptamer coated gold probe is hybridized to the DNA array.

FIG. 23: Color images of gold probe arrays prepared with different concentrations of A10-aptamer coated gold particle. The probe arrays were prepared by hybridizing A10-aptamer (SEQ ID NO: 24) coated gold particle (50 nm diameter) to a T₂₀ DNA array. Planar illumination of the glass slide with white light generates evanescent induced light scatter from the gold probes. The color images were recorded with a Zeiss Axioplan microscope equipped with a color CCD camera. The probe concentration and exposure time is listed under the image. It should be noted that the scatter color is also detectable visually with the naked eye. Scatter colors are abbreviated as follows: yg=yellow-green, and g=green

FIG. 24: Schematic illustration of human IgE detection via calorimetric scatter using anti-IgE aptamer coated gold probes. The human IgE target is incubated on the probe array, followed by a detector probe (polyclonal anti-IgE coated 50 nm gold probe) which binds to the human IgE. For detection, the substrate is illuminated with light generating optical scatter from the gold probes, which is monitored with a photosensor.

FIG. 25: Color images of human IgE assays performed on anti-IgE aptamer coated gold probe arrays. The probe arrays were incubated with different concentrations of human IgE target or an human IgG negative control samples (the target concentration is listed below the image) followed by a detector probe (polyclonal anti-IgE coated 50 nm gold probe). Planar illumination of the glass slide with white light generates evanescent induced light scatter from the gold probes. The color images were recorded with a Zeiss Axioplan microscope equipped with a color CCD camera. The observed scatter color is noted above the image.

FIG. 26. A) Images of human IgE assays on anti-IgE aptamer coated gold probe arrays recorded using the Verigene ID detection system. The Verigene ID detection system illuminates the slide with a red light emitting diode and captures an image of the slide with a monochrome photosensor. The images were taken from the same assays shown in FIG. 6 after the slide was washed to remove gold probes without target. The human IgE target concentrations are shown below each image. B) Quantitation of signal from the anti-IgE aptamer coated gold probes on the probe array. Images recorded with the Verigene ID were analyzed using Axon Genepix software. The net signal intensity was calculated by subtracting background signal from the gold probe array spots. The average net signal intensity and standard deviations from the six spots on each array are shown.

FIG. 27: The effect of dextran sulfate polymer molecular weight and intrinsic viscosity on changes in scatter color due to hybridization of DNA-modified gold nanoparticle probes to a complementary PCR product. Each sample was spotted onto an illuminated glass slide and imaged with a color CMOS sensor.

FIG. 28: The effect of dextran sulfate polymer molecular weight and intrinsic viscosity on changes in scatter color due to hybridization of DNA-modified gold nanoparticle probes to a complementary PCR product. Each sample was imaged by illuminating the sample with white light using a fiber optic probe and collecting the scattered light onto a diode array detector at 90 degrees.

FIG. 29: Changes in scatter color observed for the binding of PCR amplicons to DNA-modified gold nanoparticles using 4% dextran polymer (˜molecular weight fo 500,000) at different salt concentrations. Each sample was spotted onto an illuminated glass slide and imaged with a color CMOS sensor.

FIG. 30: Changes in scatter color observed for the binding of PCR amplicons to DNA-modified gold nanoparticles in the presence or absence 4% dextran polymer (˜molecular weight fo 500,000) or target at 0.2 M NaCl. Each sample was spotted onto an illuminated glass slide and imaged with a color CMOS sensor.

FIG. 31: Schematic of change in scatter color based on the binding of two or more DNA-modified gold nanoparticle probes to a nucleic acid target with two linker oligonucleotides composed of two gene-specific regions and one common universal region.

FIG. 32: Schematic of change in scatter color based on the binding of two or more DNA-modified gold nanoparticle probes to a nucleic acid target with two linker oligonucleotides composed of two gene-specific regions and two differing universal regions.

FIG. 33: Homogeneous detection of a nucleic acid target based on a change in scatter color using universal gold probes and intermediate oligonucleotides. A comparison of colorimetric scatter from solutions with and without gene specific intermediate oligonucleotides was performed using a single gold nanoparticle probe. A) Image of colorimetric scatter recorded using a diode array detector after illumination with white light. The scatter was recorded at 90 degrees. B) Image of colorimetric scatter recorded using a color CMOS sensor after the samples were spotted onto a glass slide. The glass slide is illuminated in the plane with white light.

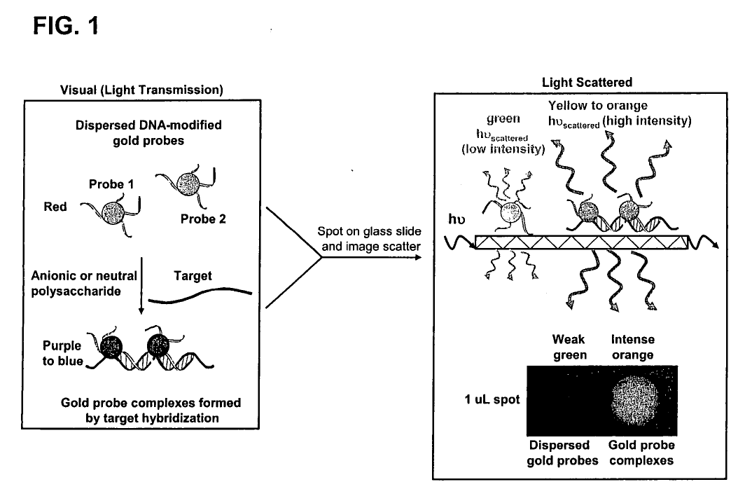

### DETAILED DESCRIPTION OF THE INVENTION

The invention provides methods for detecting protein analytes comprising the use of nucleic acid-based aptamer modified gold nanoparticles. As shown herein, nucleic acid-based aptamers, which have been developed against a variety of protein analytes for both diagnostic and therapeutic applications,^{25,34,35} can be conjugated to gold nanoparticles, and the aptamer coated gold particles can be used in conjunction with scatter-based imaging of colorimetric changes to detect protein analyte targets.

The invention also provides methods for detecting surface immobilized nucleic acid or other types of biological targets (e.g. protein or bacterial cell) by binding two or more metal nanoparticle probes to the target and measuring the color of scattered light. There are two major advantages of this detection methodology when compared to measuring total scatter from individual gold probes: 1) the gold probe complexes can be differentiated from individual gold probes non-specifically bound to the slide or to the nucleic acid target on the basis of color and intensity of scattered light, and 2) gold probe complexes scatter more light than individual gold probes enhancing signaubinding event. It is possible to use this detection methodology at the single molecule or target level since light scattered from individual metal nanoparticle probes and complexes can be detected and differentiated on the basis of the color of scattered light. There are many potential applications of this technology for detecting surface immobilized targets including, but not limited to, nucleic acid sequences from human chromosomes (e.g. in situ hybridization), human genomic DNA or RNA, bacterial DNA or RNA, viral DNA or RNA. The nucleic acid sequences may differ by only a single nucleotide such as single nucleotide polymorphisms (SNPs), or may include insertions, deletions, or sequence repeats (e.g. huntington's disease). Additionally, the surface immobilized target may be a protein, and the nanoparticle probe (a single probe with multiple binding sites) or nanoparticle probes (two or more probes with one or more binding sites) can be aptamer or antibody probes. The protein may be isolated and immobilized on the surface, or it can be part of an intact cell of an organism such as bacteria, where binding of two or more probes to the protein target can indicate the presence of a specific protein analyte or a specific organism. The surface immobilized target can be other types of biomolecules or molecules, as well, so long as the metal nanoparticle probes can bind to the molecule of interest.

In one embodiment, the probes can be used to detect nucleic acid sequences located within human chromosomes. Techniques for immobilizing human chromosomes on glass slides for in situ hybridization are well known to those of skill in the art (see for example, Ikeuchi et al., 1984, Cytogenet. Cell Genet. 38:56-61; Moorehead et al., 1960, Exp. Cell Res., 20:613-616; Priest, Medical Cytogenetics and Cell Culture, Lea and Febiger, Philadelphia, 1977; Iqbal et al., 1999, East Mediterr Health J 5:1218-24). Fluorescence probes have become the most common labeling method for in situ hybridization (FISH) (King et al., 2000, Mol Diagn 5:309-19), which has been used to diagnose chromosomal syndromes (Iqbal et al., 1999, East Mediterr Health J 5:1218-24). Fluorescence is limited by the number of bases resolvable within an in situ hybridized sample (Saracoglu et al., 2001, Cytometry 44:7-15). The methods of the invention offer single target detection capabilities based on the detection of individual nanoparticle complexes, which can be distinguished on the basis of scatter color and intensity from individual nanoparticles without separation. This aspect of the invention is critical for targets such as human chromosomes where a single gene copy may be present, and the binding of two or more nanoparticles (e.g. a nanoparticle complex) to at least a portion of the single gene copy sequence must be distinguished from individual nanoparticles non-specifically bound to the substrate or other portions of the gene sequence or chromosome. This capability enables, for example, identification of specific gene sequences (e.g. SNPs or other genetic alterations such as sequence repeats) in human chromosomal samples.

In one embodiment, the invention provides methods of detecting for the presence or absence of a single target analyte having at least two portions. In a particular embodiment, the method comprises the steps of: (a) providing a type of metal nanoparticle having a specific binding complement of a target analyte attached thereto, the specific binding complement being capable of recognizing two different segments of the target analyte; (b) forming a light scattering complex by contacting a sample believed to contain the specific binding complement with the nanoparticle and with a polysaccharide, preferably a neutral or anionic polysaccharide, under conditions effective to allow binding of the specific binding complement to two or more portions of the target analyte; (c) illuminating the light scattering complex under conditions effective to produce scattered light from said complex; and (d) detecting the light scattered by said complex as a measure of the presence of the target analyte.

In one embodiment, the invention provides methods of detecting for the presence or absence of a target analyte having at least two portions. In a particular embodiment, the method comprises the steps of: (a) providing a type of metal nanoparticle having a specific binding complement of a target analyte attached thereto, the specific binding complement being capable of recognizing two different segments of the target analyte; (b) forming a light scattering complex by contacting a sample believed to contain the specific binding complement with the nanoparticle and with a polysaccharide, preferably a neutral or anionic polysaccharide, under conditions effective to allow binding of the specific binding complement to two or more portions of the target analyte; (c) illuminating the light scattering complex under conditions effective to produce scattered light from said complex; and (d) detecting the light scattered by said complex as a measure of the presence of the target analyte.

In another embodiment, a method of the invention comprises the steps of: (a) providing at least two types of metal nanoparticles having specific binding complement of a target analyte attached thereto, the specific binding complement on each type of nanoparticles being capable of recognizing different portions of the target analyte; (b) forming a light scattering complex by contacting the target analyte with at least two types of metal nanoparticles having specific binding complements attached thereto and with a polysaccharide, preferably a neutral or anionic polysaccharide, the contacting taking place under conditions sufficient to enable binding of the specific binding complements to the target analyte; and (c) illuminating the light scattering complex under conditions effective to produce scattered light from said complex; and (d) detecting the light scattered by said complex as a measure of the presence of the target analyte.

In certain embodiments, the illumination step comprises placing at least a portion of the light scattering complex within an evanescent wave of a waveguide. In other embodiments, the detecting step comprises observing the color of scattered light from the light scattering complex and/or observing the intensity of scattered light from the light scattering complex.

In still other embodiments, a target analyte and specific binding complement can be complements of a specific binding pair. In particular embodiments, a specific binding pair can include, but is not limited to, a nucleic acid, a oligonucleotide, a polypeptide, an antibody, an antigen, a carbohydrate, a peptide nucleic acid, a protein, a peptide, an amino acid, a hormone, a steroid, a vitamin, a drug, a virus, a polysaccharide, a lipids, lipopolysaccharides, glycoproteins, lipoproteins, nucleoproteins, oligonucleotides, antibodies, immunoglobulins, albumin, hemoglobin, coagulation factors, peptide and protein hormones, non-peptide hormones, interleukins, interferons, cytokines, peptides comprising a tumor-specific epitope, cells, cell-surface molecules, microorganisms, fragments, portions, components or products of microorganisms, small organic molecules, nucleic acids and oligonucleotides, metabolites of or antibodies to any of the above substances.

In one embodiment, nucleic acids and oligonucleotides can be genes, viral RNA and DNA, bacterial DNA, fungal DNA, mammalian DNA, cDNA, mRNA, RNA and DNA fragments, synthetic oligonucleotides, modified oligonucleotides, single-stranded and double-stranded nucleic acids, natural and synthetic nucleic acids, or aptamers.

As used herein, a “target analyte” is any molecule or compound to be detected. Non-limiting examples of analytes include a nucleic acid, a oligonucleotide, a polypeptide, an antibody, an antigen, a carbohydrate, a protein, peptide nucleic acid, a peptide, an amino acid, a hormone, a steroid, a vitamin, a drug, a virus, a polysaccharide, a lipids, lipopolysaccharides, glycoproteins, lipoproteins, nucleoproteins, oligonucleotides, antibodies, immunoglobulins, albumin, hemoglobin, coagulation factors, peptide and protein hormones, non-peptide hormones, interleukins, interferons, cytokines, peptides comprising a tumor-specific epitope, cells, cell-surface molecules, microorganisms, fragments, portions, components or products of microorganisms, small organic molecules, nucleic acids and oligonucleotides, and metabolites of or antibodies to any of the above substances.

In certain embodiments of the invention, a target analyte is a nucleic acid and the specific binding complement is an oligonucleotide. In another embodiment, the target analyte is a protein or antibody and the specific binding complement is an antibody. In still another embodiment, the target analyte is a protein or antibody and the specific binding complement is a polyclonal or monoclonal antibody.

In still another embodiment, a target analyte is a gene sequence from a genomic DNA sample and the specific binding complements are oligonucleotides, the oligonucleotides having a sequence that is complementary to at least a portion of the gene sequence.

As used herein, a “single target molecule” is one target analyte that can be detected using a method of the invention. Single target molecules can be detected, for example, in a histological specimen or a metaphase spread. In certain embodiments, a single target molecule is a target nucleotide sequence that can bind to a specific binding complement of the invention. The nucleotide sequence can be a sequence of an endogenous gene (i.e. a gene found in nature, including a modified gene such as a single nucleotide polymorphism). The nucleotide sequence can also comprise an addition, deletion, transition, transversion, or modification of one or more nucleotides compared with a gene sequence of an endogenous gene, so long as the binding complement can bind to the target nucleotide sequence.

In one embodiment, the invention provides methods of homogeneous detection of target nucleic acid sequences in a sample. In one embodiment, the method comprises contacting a target nucleic acid sequence in the sample with at least one detector probe and observing a detectable change, wherein the sample comprises nucleic acid molecules of higher biological complexity relative to amplified nucleic acid molecules. Thus, the target nucleic acid sequence can be detected without enzymatic target or signal amplification. As used herein, “homogeneous detection” refers to a detection format wherein an analyte is detected by a detector probe without separating out the probe analyte complex from unbound probe.

Non-limiting examples of “enzymatic target amplification” include polymerase chain reaction (PCR), transcription mediated amplification (TMA), ligase chain reaction (LCR), and Isothermal and Chimeric Primer-Initiated Amplification of Nucleic Acid (ICAN™) (Takara Bio Inc, Japan).

A non-limiting example of “enzymatic signal amplification” include enzymatic catalysis of fluorophore labeled probes for detection of specific DNA sequences at sub-attomole levels within complex mixtures as described, for example in Hall et al., 2000, Proc. Nat. Acad. Sci. USA 97, 8272-8277, which is incorporated by reference in its entirety.

In certain embodiments, a target nucleic acid sequence of the invention can be a gene, viral RNA, viral DNA, bacterial DNA, fungal DNA, mammalian DNA, mammalian cDNA, mammalian mRNA, an oligonucleotide, or an aptamer. Preferably, the target nucleic acid sequence is DNA. In other aspects, the sample can comprise genomic DNA, genomic RNA, expressed RNA, plasmid DNA, mitochondrial or other cell organelle DNA, free cellular DNA, viral DNA or viral RNA, or a mixture of two or more of the above.

A “sample” as used herein refers to any quantity of a substance that comprises an analyte and that can be used in a method of the invention. For example, the sample can be a biological sample or can be extracted from a biological sample derived from humans, animals, plants, fungi, yeast, bacteria, viruses, tissue cultures or viral cultures, or a combination of the above. They may contain or be extracted from solid tissues (e.g. bone marrow, lymph nodes, brain, skin), body fluids (e.g. serum, blood, urine, sputum, seminal or lymph fluids), skeletal tissues, or individual cells. Alternatively, the sample can comprise purified or partially purified nucleic acid molecules and, for example, buffers and/or reagents that are used to generate appropriate conditions for successfully performing a method of the invention. A sample can be prepared and used in a method of the invention from a swab (such as a cotton or buccal swab), culture, cellular extract, or lysed cells.

In a particular embodiment, nucleic acid molecules in a sample are of higher biological complexity than amplified nucleic acid molecules. One of skill in the art can readily determine the biological complexity of a target nucleic acid sequence using methods as described, for example, in Lewin, GENE EXPRESSION 2, Second Edition: Eukaryotic Chromosomes, 1980, John Wiley & Sons, New York, which is hereby incorporated by reference.

Hybridization kinetics are absolutely dependent on the concentration of the reaction partners, i.e. the strands that have to hybridize. In a given quantity of DNA that has been extracted from a cell sample, the amount of total genomic, mitochondrial (if present), and extra-chromosomal elements (if present) DNA is only a few micrograms. Thus, the actual concentrations of the reaction partners that are to hybridize will depend on the size of these reaction partners and the complexity of the extracted DNA. For example, a target sequence of 30 bases that is present in one copy per single genome is present in different concentrations when comparing samples of DNA from different sources and with different complexities. For example, the concentration of the same target sequence in 1 microgram of total human DNA is about 1000 fold lower than in a 1 microgram bacterial DNA sample, and it would be about 1,000,000 fold lower than in a sample consisting in 1 microgram of a small plasmid DNA.

The high complexity (1×10⁹ nucleotides) of the human genome demands an extraordinary high degree of specificity because of redundancies and similar sequences in genomic DNA. For example, to differentiate a nucleic acid sequence with 25meric oligonucleotides from the whole human genome requires a degree of specificity with discrimination ability of 40,000,000:1. In addition, since the wild type and mutant targets differ only by one base in 25mer capture sequence, it requires distinguishing two targets with 96% homology for successful genotyping. The methods of the invention surprisingly and unexpectedly provide efficient, specific and sensitive homogeneous detection of a target nucleic acid molecule having high complexity compared with amplified nucleic acid molecules.

The biological complexity of target nucleic acid molecules in a sample derived from human tissues is on the order of 1,000,000,000, but may be up to 10 fold higher or lower for genomes from plants or animals. Preferably, the biological complexity of a sample is about 50,000 to 5,000,000,000. More preferabley, the biological complexity is about 1,000,000-6,000,000. Most preferably, the biological complexity is about 1,000,000,000.

In certain other embodiments, a detector probe of the invention is a nanoparticle, preferably a metallic nanoparticle and more preferably a gold nanoparticle, bound to one or more oligonucleotides that have a sequence that is complementary to at least part of the target nucleic acid sequence.

In other embodiments, a homogeneous detection method of the invention comprises contacting a target nucleic acid with one or more detector probes in the presence of a neutral or anionic polysaccharide, preferably an anionic polysaccharide, more preferably dextran sulfate, thereby forming a light scattering complex, and wherein the light scattering complex is illuminated under conditions effective to produce scattered light from said complex, from which the presence of the target nucleic acid sequence can be detected. In this embodiment, the detection probes are nanoparticles, preferably metallic nanoparticles and more preferably gold nanoparticles, each bound to an oligonucleotide having a sequence that is complementary to a different portion of the target nucleic acid sequence. Scattered light can be detected as described herein to detect the presence of the target nucleic acid sequence.

In one embodiment, the methods of the invention involve detecting a change in the scattering of light directed into a waveguide, the change in scattering being the result of the formation of analyte mediated nanoparticle probe complexes in the presence of a neutral or anionic polysaccharide, which are formed on a waveguide surface, or formed in solution and spotted onto the surface, such that at least a portion of the sample is within the penetration depth of an evanescent wave. A waveguide refers to a two dimensional total internal reflection (TIR) element that provides an interface capable of internal reference at multiple points, thereby creating an evanescent wave that is substantially uniform across all or nearly the entire surface. The two dimensional waveguide can be planar or curve linear and can assume the shape of a cuvette, a rod or a plate. The waveguide can be comprised of transparent material such as glass, quartz, plastics such as polycarbonate, acrylic, or polystyrene. Preferably, the waveguide is a planar waveguide, for example, a glass slide.

The formation of nanoparticle probe complexes in a method of the invention can occur in solution by the specific binding interaction of two or more nanoparticle labels to a target analyte (or alternatively one nanoparticle binding at multiple sites on a single target) in the presence of a neutral or anionic polysaccharide. A waveguide can be contacted with the solution so that a change in scatter due to formation of nanoparticle probe complexes can be measured. When the nanoparticle probe complexes are comprised of metal nanoparticle probes, changes in the surface plasmon band frequency and intensity may be measured in the form of color and intensity of scattered light. In one embodiment of the invention, the sample can be placed within the penetration depth of an evanescent field (e.g. spotted from solution), and the light scattering from the sample can be measured without the need of capture probes. Alternatively, the sample can be spotted onto the waveguide and subsequently dried. This procedure can enhance detection sensitivity and provide a permanent test record.

The glass or other types of surfaces used for waveguides can be modified with any of a variety of functional groups (e.g. —COOH, —NH₂, —OH, etc.), including specific binding members such as haptens or oligonucleotide sequences. In this embodiment, a neutral or anionic polysaccharide (e.g. dextran sulfate) enables binding of one or more suitably functionalized metal nanoparticles to adjacent regions of a target analyte in a homogeneous format. An example is the binding of two DNA-modified metallic nanoparticles to adjacent regions of a complementary nucleic acid sequence (e.g. PCR amplicon) in solution.

As described herein, in experiments with PCR amplicons or genomic DNA, >2% v/v dextran sulfate (average molecular weight of 500,000) enabled binding of two or more DNA-modified gold probes to a complementary target sequence leading to a detectable change in colorimetric scatter when samples were spotted onto an illuminated glass waveguide. Using 2-5% v/v dextran sulfate exhibited similar detectable changes in colorimetric scatter for target analytes, while 1% v/v dextran sulfate did not result in a change in colorimetric scatter under these conditions. The dextran sulfate polymer excludes volume in the solution, and the rate constant of DNA renaturation has been shown to be proportional to the concentration of polymer and to be proportional to the intrinsic viscosity of the polymers, and also will depend on the chemical composition of the polymer (Wetmur et. al, Biopolymers 14, 2517 (1975)). Wetmur and co-workers defined the rate acceleration of DNA renaturation, R, using the following equation [1]:
R=e^{-0.4β[n]c} [1]
where n is the intrinsic viscosity of the polymer, c is the weight concentration of the polymer, and β is related to the chemical composition of the polymer.

This equation predicts that a plot of the logarithm of the DNA renaturation rate constant versus weight concentration of an added polymer should be a straight line. Although a direct measurement of rate constant was not performed, our results are in general agreement since increased concentrations of dextran sulfate enhance the color change due to facilitated nanoparticle probe-target complex formation.

This equation also predicts that a plot of the DNA renaturation rate constant versus intrinsic viscosity of the polymer should also produce a straight line when plotted. Wetmur and coworkers tested this equation by monitoring DNA renaturation rate in the presence of dextran polymers of different molecular weight (40,000-2,000,000), where the intrinsic viscosity of the polymer increases with molecular weight. Using the same concentration of polymer (7.5%), Wetmur and coworkers demonstrated that increasing the molecular weight of the dextran polymer (and therefore in the intrinsic viscosity) increased DNA renaturation rates according to the equation. The formation of DNA-modified nanoparticle probe—nucleic acid target complexes follows a similar trend, where an increased change in scatter color is observed as the molecular weight of the dextran sulfate polymer is increased at a given polymer concentration, which corresponds to an increase in intrinsic viscosity of the solution (see Examples below).

Furthermore, Wetmur and coworkers demonstrated that the β value, and therefore the renaturation rate, changes depending on the chemical composition of the polymer. Measured β values and therefore renaturation rates were higher for dextran sulfate polymers when compared to dextran polymers of the same molecular weight in their study. Studies performed on the formation of nanoparticle probe—target complexes demonstrate that either dextran sulfate or dextran polymers may be used to enhance formation of the nanoparticle probe complexes and therefore a color change (see examples below). The use of dextran polymers is facilitated by the addition of NaCl to the buffer (0.2 M NaCl works best). The previously demonstrated experiments as well as the examples below with dextran sulfate polymers (sodium salts) at 4% w/w in solution are estimated to have ˜0.2 M Na ion concentration in solution (based on an estimate of 2.5 sulfates/monomer and one sodium counter ion for each sulfate using a molecular weight of 500,000) and therefore the addition of NaCl to the dextran provides a roughly equivalent amount of NaCl in the sample.

It can be concluded that any polymer or other type of molecule that excludes volume, increases the intrinsic viscosity, or in the case of DNA increases the rate of DNA renaturation of the solution, could be used in the methods of the invention. In addition to double stranded nucleic acids, dextran sulfate (average molecular weight of 500,000) enhances binding of polyclonal antibody coated gold probes to a protein-based target leading to a detectable change in colorimetric scatter when samples are spotted onto an illuminated glass waveguide.

Thus, in one embodiment of the invention, 0.1-10% v/v of a neutral or anionic polysaccharide, such as the anionic polysaccharide dextran sulfate having an average molecular weight of 500,000, can be used to promote the formation of probe-target complexes. Preferably, 2-5% v/v of a neutral or anionic polysaccharide, such as the anionic polysaccharide dextran sulfate with an average molecular weight of 500,000, is used for probe-target binding. Alternatively, more than 10% neutral or anionic polysaccharide, such as the anionic polysaccharide dextran sulfate, can be used in the methods of the invention.

The average molecular weight range of dextran sulfate used in the Examples described below ranged from ˜10,000 to about ˜1,000,000. However, dextran sulfate and derivatives thereof can be produced at a variety of molecular weights. At the top end, the average molecular weight exceeds 1 million. On the other end, molecular weights of dextran sulfate can be less than 100,000. A preferred molecular weight range for the methods of the invention is between 10,000 and 2,000,000. A more preferred molecular weight range for the methods of the invention is between 500,000 and 2,000,000. The most preferred molecular weight range is 500,000-1,000,000 with an average molecular weight between 500,000 and 1,000,000.

Dextran sulfate sodium salt is a polyanionic derivative of dextran. It has been demonstrated previously that both neutral and anionic dextran polymers accelerate DNA renaturation (Wetmur et. al, Biopolymers, 14,2517 (1975)). Therefore, both neutral and polyanionic polysaccharides can be used in the methods of the invention to enable probe-target analyte complex formation. Furthermore, natural mucopolysaccharides (e.g. chondroitin sulfate and dermatan sulfate) are similar in chemical composition and can also be used in methods of the invention to enable probe—target analyte complex formation. In addition, other natural or synthetic polymers that are neutral, anionic, or cationic, or a mixture thereof, also can be used in the methods of the invention. A non-limiting example is CTAB (cetyltrimethylammonium bromide), which also has been shown to accelerate DNA renaturation rates.

In one embodiment, metallic nanoparticles are employed as the light-scattering label in a method of the invention. Such labels cause incident light to be scattered elastically, i.e. substantially without absorbing light energy. Suitable but non-limiting nanoparticles and methods for preparing such nanoparticles are described in U.S. Pat. No. 6,506,564, issued Jan. 14, 2003; U.S. Ser. No. 09/820,279, filed Mar. 28, 2001; U.S. Ser. No. 008,978, filed Dec. 7, 2001; U.S. Ser. No. 10/125,194, filed Apr. 18, 2002; U.S. Ser. No. 10/034,451, filed Dec. 28, 2001; International application no. PCT/US01/10071, filed Mar. 28, 2001; International application no. PCT/US01/46418, filed Dec. 7, 2001; and International application no. PCT/US02/16382, filed May 22, 2002, all which are incorporated by reference in their entirety. Metal nanoparticles >30 nm diameter are preferred for homogenous detection of probe-target analyte complexes on an illuminated waveguide. Metal nanoparticles >30 nm diameter are known to scatter light with high efficiency, where the scattering intensity scales with the sixth power of the radius for individual particles. Further, the surface plasmon band frequency of metal nanoparticles, which leads to the absorbance and scattering of specific wavelengths of light, is dependent on particle size, chemical composition, particle shape, and the surrounding medium, such that a decrease in interparticle distance between two or more metal nanoparticles results in changes in the surface plasmon band frequency and intensity. For example, when two metal nanoparticle particles with specific binding members bind to adjacent regions of a target analyte, a change in the surface plasmon band frequency occurs leading to a change in solution color. Metal nanoparticles in the size range of 40-80 nm diameter are most preferred since monodisperse particles (<15% CV) can be synthesized, and the changes in the color and intensity of scattered light can be monitored visually or with optical detection instrumentation on an illuminated waveguide. A variety of metal nanoparticle compositions also could be used in the reported invention including gold, silver, copper, and other metal particles well known in the art or alloy or core-shell particles. For example, a core-shell particle can be a nanoparticle having a metal or non-metal (e.g. silica or polystyrene) core coated with a shell of metal. Such core-shell particles are described, for example, in Halas et al., 1999, Applied Physics Letters 75:2197-99 and Halas et al., 2001, J of Phys Chem. B 105:2743, which is incorporated by reference herein in its entirety. In one embodiment, other types of metal nanostructures that have a surface plasmon band can be used in the methods of the invention. The most preferred particle composition is gold since it is highly stable and can be derivatized with a variety of biomolecules. The most preferred particle and size range is 40-80 nm diameter gold particles.

When using dextran sulfate to drive the formation of nanoparticle probe-target analyte complexes, the preferred detection embodiment is an illuminated waveguide, which enables the monitoring of scattered light from the complexes within the penetration depth of the evanescent field. In addition to high detection efficiency associated with monitoring nanoparticle scatter, which is well known in the art, the formation of metal nanoparticle probe-target complexes not only leads to a shift in color, but also provides a substantial increase in the intensity of light scattered when compared to an uncomplexed metal nanoparticle probe.

Unlike previously reported systems, this enables homogeneous detection of target analytes in the presence of an excess of nanoparticle probes. An example is two 50 nm gold probes bound to a DNA target, where a visually detectable color change is observed on the waveguide in the presence of up to 20 fold excess of unbound gold nanoparticle probes after the sample is dried onto the waveguide (note that the sample may not be fully dried as dextran sulfate retains some moisture under some conditions), without removing the excess unbound gold nanoparticle (i.e. homogeneous reaction). As a result, homogeneous detection of target analyte can be driven with an excess of nanoparticle probe, and in conjunction with dextran sulfate enables femtomolar concentrations of target analyte (e.g. specific genomic DNA sequences) to be detected with picomolar concentrations of 50 nm diameter gold probe.

In addition, the detectable probe/target ratio can be increased substantially by using more than two probes that bind to a target analyte. By binding four 50 nm gold probes to adjacent regions of a DNA target in the homogeneous assay, over 200 fold excess of gold nanoparticle probe can be used in the methods of the invention, and a change in colorimetric scatter is still detectable on an illuminated waveguide. By using an excess of probe to target, significantly lower concentrations of target analyte can be detected with the methods of the invention either visually or with optical detection instrumentation.

The Examples described herein demonstrate use of either two probes or four probes complexed to a target analyte. In one embodiment, a larger number of probes can be used in methods of the invention to bind to a target analyte to achieve even greater detection sensitivities and larger probe/target ratios. In another embodiment of the invention, intermediate oligonucleotides which contain a sequence portion complementary to the target analyte, and a second sequence portion complementary to a probe, may be bound to the target analyte, followed by binding of the nanoparticle probe. By designing intermediate oligonucleotides that bind to different portions of a target analyte but have a common recognition sequence for a nanoparticle, two or more nanoparticle probes can be bound to a target analyte using a single nanoparticle probe with multiple intermediate oligonucleotides. Non-evanescent scatter-based detection methods well known in the art are compatible with the methods of the invention for forming metal nanoparticle probe complexes in the presence of neutral or anionic polysaccharides. Other methods such as light absorbance, light transmission, light reflectance, surface enhance raman scattering, electrical, as well as other detection methods well known in the art for detecting nanoparticle probe complexes also can be used.

In another embodiment of the invention, the waveguide includes at least one or more discrete regions that contain the same or different specific binding members or capture probes attached thereto to immobilize one or more different target analytes. Preferably the discrete region is in the form of a small dot or spot. Other sizes and configurations are possible and are within the scope of the invention. Alternatively, the waveguide may include a plurality of arrayed discrete regions to target different portions of a single analyte or multiple different analytes.

As defined here, the “specific binding member” means either member of a cognate binding pair. A “cognate binding pair,” as defined herein, is any ligand-receptor combination that will specifically bind to one another, generally through non-covalent interactions such as ionic attractions, hydrogen bonding, Vanderwaals forces, hydrophobic interactions and the like. Exemplary cognate pairs and interactions are well known in the art and include, by way of example and not limitation: immunological interactions between an antibody or Fab fragment and its antigen, hapten or epitope; biochemical interactions between a protein (e.g. hormone or enzyme) and its receptor (for example, avidin or streptavidin and biotin), or between a carbohydrate and a lectin; chemical interactions, such as between a metal and a chelating agent; and nucleic acid base pairing between complementary nucleic acid strands; a peptide nucleic acid analog which forms a cognate binding pair with nucleic acids or other PNAs. Nucleic acid will be understood to include 2′-deoxyribonucleic acid (DNA) as well as ribonucleic acid (RNA) when stability permits. Preparation of antibody and oligonucleotide specific binding members is well known in the art.

The analyte-probe complexes of the invention can be observed directly on a waveguide without specific attachment (e.g. no interaction with the surface, ionic interaction with the surface, or van der Waals interaction with the surface), or with non-covalent attachment. Alternatively, the specific binding members can be covalently attached to the waveguide through chemical coupling means known in the art. The reactive surface can be derivatized directly with a variety of chemically reactive groups which then, under certain conditions, form stable covalent bonds with the applied specific binding member. The application of the capture specific binding members onto the reactive surface can be accomplished by any convenient means, such as manual use of micropipets or microcapillary tubes or by automated methods such as positive displacement pumps, X-Y positioning tables, and/or ink jet spraying or printing systems and the like. Any suitable density (quantity per unit area) of capture specific binding members on the reactive surface can be used.

In addition to immobilization of capture specific binding member to the reactive surface, the reactive surface is preferably treated so as to block non-specific interactions between the reactive surface and analyte binding members in a fluid sample that is to be tested. In the case of a protein specific binding member (e.g. antigen, antibody or PNA) on the reactive surface, the blocking material should be applied after immobilization of the specific binding member. Suitable blocking materials include, without limitation, casein, zein, bovine serum albumin (BSA), detergents and long-chain water soluble polymers. In the case of a nucleic acid SBM, the blocking material can be applied before or after immobilization of the SBM. Suitable blockers include those described above as well as 0.5% sodium dodecyl sulfate (SDS) and Denhardt's solution.

It should be understood that the first specific binding member can be specific for the analyte through the intermediary of additional cognate pairs if desired. For example, an oligonucleotide specific binding member might be biotinylated and attached to the reactive surface via a biotin-avidin cognate binding pair. Such an attachment is described, for example, by Hansen in EP 0 139 489 (Ortho), which is incorporated by reference in its entirety. Similarly, an oligonucleotide might be attached to the reactive surface through a mediator probe as disclosed, for example, by Stabinsky in U.S. Pat. No. 4,751,177 (Amgen), which is incorporated by reference in its entirety.

In the present invention, the nanoparticle can be attached to a first specific binding member of a second cognate binding pair. The second specific binding pair member can be referred to as a “label specific binding member” and the complex of the nanoparticle and label specific binding member is referred to as “label conjugate” or just “conjugate”. For a direct sandwich assay format, the label specific binding member is specific for a second epitope on the analyte. This permits the analyte to be “sandwiched” between the capture specific binding member and the label specific binding member. In an indirect sandwich assay format, the label specific binding member is specific for a site or reporter group that is associated with the analyte. For example, once an antigenic analyte is captured, a biotinylated antibody can be used to “sandwich” the analyte, and biotin-specific label specific binding member is used. This indirect sandwich format is also useful for nucleic acids. In this case the capture specific binding member can be an oligonucleotide complementary to the target and the target can contain a specific binding reporter molecule (e.g. biotin or a hapten, typically incorporated via an amplification procedure such as LCR or PCR) and the label specific binding member can be chosen to be specific for the reporter group.

The label specific binding member can be specific for its respective partner (analyte or first specific binding member, depending on the format) through intermediary cognate pairs, as was the case with the capture specific binding member. For example, if the analyte is an oligonucleotide such as an amplification product bearing a hapten reporter group, a sandwich assay format might include a nanoparticle conjugated to antihapten antibody. Thus, the label specific binding member is specific for the analyte via the hapten-antihapten cognate binding pair.

Regardless of the assay format, the label specific binding member can attach to the nanoparticle to form the conjugate. As with capture specific binding member, the label specific binding member can be covalently bonded to the nanoparticle. Physical adsorption of label specific binding member onto nanoparticles is also suitable. In such case, the attachment need only be strong enough to withstand the subsequent reaction conditions without substantial loss of nanoparticle, e.g. from washing steps or other fluid flow.

A large number of strategies suitable for coupling the nanoparticle and the label specific binding member exist. See, for instance, U.S. Pat. No. 6,506,564, issued Jan. 14, 2003; U.S. Ser. No. 09/820,279, filed Mar. 28, 2001; U.S. Ser. No. 008,978, filed Dec. 7, 2001; U.S. Ser. No. 10/125,194, filed Apr. 18, 2002; U.S. Ser. No. 10/034,451, filed Dec. 28, 2001; International application no. PCT/US01/10071, filed Mar. 28, 2001; International application no. PCT/US01/46418, filed Dec. 7, 2001; and International application no. PCT/US02/16382, filed May 22, 2002, which are incorporated by reference in their entirety.

Scattered light can be detected visually or by photoelectric means. For visual detection, the observer visually determines whether or not scattering has occurred at a discrete region. For instance, scattering is observed when the discrete region appears brighter than the surrounding background or a control spot that contains uncomplexed particles located at an adjacent region. Alternatively, the observer can determine what color of light is scattered at a discrete region. For instance, a scatter color of orange at a discrete region of interest can be compared to the surrounding background or to a control spot containing uncomplexed particles that scatters no light or weak green light depending on particle size located at an adjacent region. If there are numerous discrete regions, a photoelectric detection system is preferred. Photoelectric detection systems include any system that uses an electrical signal which is modulated by the light intensity and/or frequency at the discrete region.

There are a number of avenues with different modes of illumination and imaging that are demonstrated herein for the detection of gold nanoparticle complexes on transparent substrates for the purposes of biomolecule or molecular detection. In the first method, planar illumination of a transparent substrate with white light generates an evanescent wave on the slide surface, and the light scattered from samples on the substrate is collected with a monochrome photosensor (e.g. CMOS or CCD). In the second method, planar illumination of a transparent substrate with white light generates an evanescent wave on the slide surface, and the light scattered from samples on the substrate is collected with a color photosensor (e.g. CMOS or CCD). In the third method, planar illumination of a transparent substrate with a specific wavelength of light generates an evanescent wave at the slide surface, and the light scattered from samples on the substrate is collected with a monochrome or color photosensor. An alternative method is planar illumination of a transparent substrate with white light, which generates an evanescent wave at the slide surface, and the light scattered from samples on the substrate is filtered with a specific wavelength filter and collected onto a monochrome photosensor. In addition, the light scattered from probe complexes formed in the presence of neutral or anionic polysaccharide can be monitored using non-evanescent scattering techniques. The light scattered from probe complexes also may be detected using a diode array detector.

Unless otherwise required by context, singular terms used herein shall include pluralities and plural terms shall include the singular.

### EXAMPLES

The invention is demonstrated further by the following illustrative examples. The examples are offered by way of illustration and are not intended to limit the invention in any manner. In these examples all percentages are by weight if for solids and by volume if for liquids, and all temperatures are in degrees Celsius unless otherwise noted.

### Example 1

### Preparation of Nanoparticle-Oligonucleotide Conjugate Probes

In this Example, a representative nanoparticle-oligonucleotide conjugate detection probe was prepared for the use in the detection of APC, Factor V Leiden gene, or mecA gene targets.

(a) Preparation of 15 nm Diameter Gold Nanoparticles

Gold colloids (˜15 nm diameter) were prepared by reduction of HAuCl₄ with citrate as described in Frens, 1973, Nature Phys. Sci., 241:20-22 and Grabar, 1995, Anal. Chem. 67:735. Briefly, all glassware was cleaned in aqua regia (3 parts HCl, 1 part HNO₃), rinsed with Nanopure H₂O, then oven dried prior to use. HAuCl₄ and sodium citrate were purchased from Aldrich Chemical Company. Aqueous HAuCl₄ (1 mM, 500 mL) was brought to reflux while stirring. Then, 38.8 mM sodium citrate (50 mL) was added quickly. The solution color changed from pale yellow to burgundy, and refluxing was continued for 15 min. After cooling to room temperature, the red solution was filtered through a Micron Separations Inc. 0.2 micron cellulose acetate filter. Au colloids were characterized by UV-vis spectroscopy using a Hewlett Packard 8452A diode array spectrophotometer and by Transmission Electron Microscopy (TEM) using a Hitachi 8100 transmission electron microscope.

(b) 30, 40, and 50 nm Diameter Gold Nanoparticles

Solutions of 30, 40, and 50 nm diameter gold particles were purchased from Ted Pella, Inc. for the described experiments.

(c) Synthesis of Steroid Disulfide Modified Oligonucleotides (SDO)

Oligonucleotides complementary to segments of the APC gene DNA sequence or Factor V Leiden gene DNA sequence were synthesized on a 1 micromole scale using a Applied Biosystems Expedite 8909 DNA synthesizer in single column mode using phosphoramidite chemistry. Eckstein, F. (ed.) Oligonucleotides and Analogues: A Practical Approach (IRL Press, Oxford, 1991). All synthesis reagents were purchased from Glen Research or Applied Biosystems. Average coupling efficiency varied from 98 to 99.8%, and the final dimethoxytrityl (DMT) protecting group was removed from the oligonucleotides so that the steroid disulfide phosphoramidite could be coupled.

To facilitate hybridization of the probe sequence with the target, a deoxyadenosine oligonucleotide (dA₂₀) or deoxyadenosine oligonucleotide-polyethylene glycol (dA₁₅-PEG) was included on the 5′ end in the probe sequence as a spacer.

To generate 5′-terminal steroid-cyclic disulfide oligonucleotide derivatives (see Letsinger et al., 2000, Bioconjugate Chem. 11:289-291 and PCT/US01/01190 (Nanosphere, Inc.), the disclosure of which is incorporated by reference in its entirety), the final coupling reaction was carried out with a cyclic dithiane linked epiandrosterone phosphoramidite on Applied Biosystems automated Expedite 8909 synthesizer, a reagent that prepared using trans 1,2-dithiane-4,5-diol, epiandrosterone and p-toluenesulphonic acid (PTSA) in presence of toluene. The phosphoramidite reagent may be prepared as follows: a solution of epiandrosterone (0.5 g), trans 1,2-dithiane-4,5-diol (0.28 g), and p-toluenesulfonic acid (15 mg) in toluene (30 mL) was refluxed for 7 h under conditions for removal of water (Dean Stark apparatus); then the toluene was removed under reduced pressure and the reside taken up in ethyl acetate. This solution was washed with 5% NaHCO₃, dried over sodium sulfate, and concentrated to a syrupy reside, which on standing overnight in pentane/ether afforded a steroid-dithioketal compound as a white solid (400 mg); Rf (TLC, silica plate, ether as eluent) 0.5; for comparison, Rf values for epiandrosterone and 1,2-dithiane-4,5-diol obtained under the same conditions are 0.4, and 0.3, respectively. The compound was purified by column chromatography. Subsequently, recrystallization from pentane/ether afforded a white powder, mp 110-112° C.; ¹H NMR, δ 3.6 (1H, C³OH), 3.54-3.39 (2H, m 2OCH of the dithiane ring), 3.2-3.0 (4H, m 2CH₂S), 2.1-0.7 (29H, m steroid H); mass spectrum (ES⁺) calcd for C₂₃H₃₆O₃S₂ (M+H) 425.2179, found 425.2151. Anal. (C₂₃H₃₇O₃S₂) S: calcd, 15.12; found, 15.26. To prepare the steroid-disulfide ketal phosphoramidite derivative, the steroid-dithioketal (100 mg) was dissolved in THF (3 mL) and cooled in a dry ice alcohol bath. N,N-diisopropylethylamine (80 μL) and β-cyanoethyl chlorodiisopropylphosphoramidite (80 μL) were added successively; then the mixture was warmed to room temperature, stirred for 2 h, mixed with ethyl acetate (100 mL), washed with 5% aq. NaHCO₃ and with water, dried over sodium sulfate, and concentrated to dryness. The residue was taken up in anhydrous acetonitrile and then dried under vacuum; yield 100 mg; ³¹P NMR 146.02. The epiandrosterone-disulfide linked oligonucleotides were synthesized on Applied Biosystems Expedite 8909 gene synthesizer without final DMT removal. After completion, epiandrosterone-disulfide linked oligonucleotides were deprotected from the support under aqueous ammonia conditions and purified on HPLC using reverse phase column.

Reverse phase HPLC was performed with a Dionex DX500 system equipped with a Hewlett Packard ODS hypersil column (4.6×200 mm, 5 mm particle size) using 0.03 M Et₃NH⁺ OAc⁻ buffer (TEAA), pH 7, with a 1 mL/min. gradient of 95% CH₃CN/5% TEAA. The flow rate was 1 mL/min. with UV detection at 260 nm. Preparative HPLC was used to purify the DMT-protected unmodified oligonucleotides. After collection and evaporation of the buffer, the DMT was cleaved from the oligonucleotides by treatment with 80% acetic acid for 30 min. at room temperature. The solution was then evaporated to near dryness, water was added, and the cleaved DMT was extracted from the aqueous oligonucleotide solution using ethyl acetate. The amount of oligonucleotide was determined by absorbance at 260 nm, and final purity assessed by reverse phase HPLC.

(d) Attachment of SDOs to 30, 40, or 50 nm Diameter Gold Particles

Solutions of 30, 40, or 50 nm diameter gold particle were used as delivered from Ted Pella, Inc. The gold nanoparticle probes were prepared by loading the gold particles with steroid disulfide modified oligonucleotides using a modification of a previously developed literature procedure¹⁵. Briefly, 8 nmol of SDO was added per 3 mL of gold nanoparticle and incubated for 15 hours at room temperature. After 24 hours, aqueous sodium dodecyl sulfate (SDS, 10% by weight) was added to the solution (final concentration: 0.01%). Then, aqueous 2 M NaCl was added to a final concentration of 0.1 M NaCl. After standing for 24 additional hours, the NaCl concentration was increased to 0.2 M. This was repeated the following day to bring the NaCl concentration of the probe solution to 0.3 M. After 24 additional hours, the SDO-gold nanoparticle conjugates were isolated with a Beckman Coulter Microfuge 18 by centrifugation (5000 rpm for 25 minutes for 30 nm, 3000 rpm for 15 minutes for 40 nm, 3000 rpm for 15 minutes for 50 nm). After centrifugation, a dark red gelatinous residue remained at the bottom of the eppendorf tube. The supernatant was removed, and the conjugates were washed (2×) with 0.1 M NaCl, 10 mM phosphate (pH 7) (original colloid volume) and redispersed in 20 mM Tris HCL (pH 7).

The following nanoparticle-oligonucleotide conjugates specific for segments of the APC gene of the human genome were prepared in this manner:

Probe APC 1-WT:

(SEQ ID NO:1)

gold-S′-5′-[a₂₀-gcagaaataaaag-3′]ₙ
Probe APC 1-MUT:

(SEQ ID NO:2)

gold-S′-5′-[a₂₀-gcagaaaaaaaag-3′]ₙ

Probe APC 2:

(SEQ ID NO:3)

gold-S′-5′-[a₂₀-aaaagattggaacta-3′]ₙ
Probe Factor V 1-WT:

(SEQ ID NO:4)

gold-S′-5′-[a₂₀-tattcctcgcc-3′]ₙ
Probe Factor V 1-MUT:

(SEQ ID NO:5)

gold-S′-5′-[a₂₀-attccttgcc-3′]ₙ
Probe Factor V 2:

(SEQ ID NO:6)

gold-S′-5′-[a₂₀-ctgctcttacagattagaag-3′]ₙ

S′ indicates a connecting unit prepared via an epiandrosterone disulfide group; n indicates that a number of oligonucleotides are attached to each gold nanoparticle.

TABLE 1

Sequences of synthetic targets and PCR amplicons
probes used for assay development.

| | | |
|---|---|---|
| MTHFR gene | 5′tattggcaggttaccccaaagg | SEQ ID NO:7 |
| 119 PCR | ccaccccgaagcagggagctttga | |
| amplicon | ggctgacctgaagcacttgaagga | |
| | gaaggtgtctgcgggagccgattt | |
| | catcatcacgcagctttt | |
| | ctttgag3′ | |
| APC gene 78 | 5′cgctcacaggatcttcagctga | SEQ ID NO:8 |
| base | cctagttccaatcttttcttttat | |
| sequence- | ttctgctatttgcagggtattagc | |
| Wild type | agaatctg3′ | |
| (1) | | |
| APC gene 78 | 5′cgctcacaggatcttcagctga | SEQ ID NO:9 |
| base | cctagttccaatcttttctttttt | |
| sequence- | ttctgctatttgcagggtattagc | |
| Mutant (2) | agaatctg3′ | |
| Factor V | 5′gacatcgcctctgggctaatag | SEQ ID NO:10 |
| Leiden 99 bp | gactacttctaatctgtaagagca | |
| PCR product | gatccc | |

### Example 2

### The Use of Dextran Sulfate as a Hybridization Facilitator for Detection of Double Stranded Nucleic Targets with DNA-Modified Gold Nanoparticle Probes

Metallic nanoparticles (30-120 nm diameter) have the potential to be used in homogeneous scatter-based detection of specific nucleic acid sequences in PCR amplicons or even genomic DNA samples, which has never been demonstrated. Light scattered from gold or silver particle increases with the sixth power of the radius, and a specific color of scattered light is emitted based on the particle composition, size, and shape.^{36,37} For example, 30-60 nm diameter gold particles scatter green colored light based on the surface plasmon resonance frequency, and light scattered from gold particles in this size range can be detected at picomolar—femtomolar particle concentrations using detection instrumentation that monitors non-evanescent light scattering.^{36,37} In homogeneous reactions, the color of absorbed or scattered light changes if the particles are brought into close proximity. Therefore, it is highly desirable to use the light scattering capabilities of metallic nanoparticles to detect specific biomolecules.

The use of metallic nanoparticles in homogeneous biomolecule detection assays has been limited to date because conditions used for homogeneous detection of bioanalytes (e.g. DNA, proteins) have not been directly applicable to gold nanoparticle probes. For example, it was previously demonstrated that a snap freeze was required to bind 15 nm diameter gold particle probes labeled with DNA to a denatured double stranded nucleic acid. However, freezing is not amenable to automation and promotes mismatch formation at the lower temperature, and therefore, it is not suitable for more complex target analyte samples such as genomic DNA where a large number of non-target sequences are present, or for protein-based reactions where continuous freeze-thaw reactions may damage the protein or antibody. Even with longer PCR amplicons or certain sequences, freezing can lead to intrastrand base-pairing preventing nanoparticle probe—analyte target complex formation.

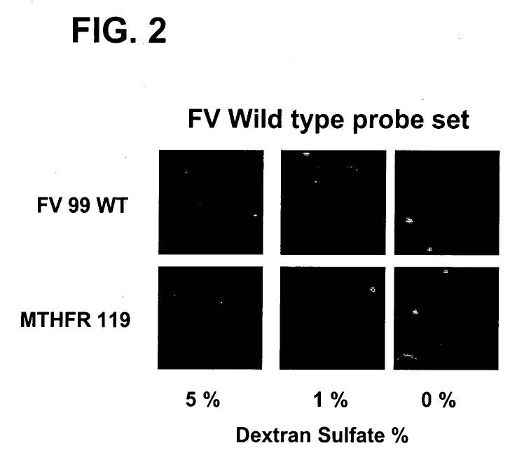

Previous studies have demonstrated that nucleic acid hybridization reactions are accelerated by polysaccharides such as the anionic polysaccharide dextran sulfate. Dextran sulfate was tested as a facilitator of gold probe—target analyte complex formation using 40 nm diameter gold probes labeled with nucleic acids (SEQ ID NO: 4 and 6) that are complementary to a Factor V Leiden PCR amplicon (SEQ ID NO: 10). For testing, 5 ul of each probe (705 pM), 5 uL of PCR amplicon (SEQ ID NO: 10), 1 ul of 25 mM MgCl₂, and 4 uL of x % dextran sulfate were added to vary the dextran sulfate concentration. Five microliters of an MTHFR 119 bp PCR amplicon (SEQ ID NO: 7) was used as a negative control for this set of experiments. The solutions were hybridized for 15 minutes at room temperature, and then 1 ul of the sample was spotted onto a poly-L-lysine glass slide and imaged wet using a color CMOS, FIG. 2. As expected, no color changes were observed under standard hybridization conditions indicating that no gold probe—target analyte complexes were formed. With the addition of 5% dextran sulfate, light scattered from the FV 99 target solution exhibited a highly intense orange color, while significantly less intense green light was scattered from the remaining target and control solutions. This indicates that dextran sulfate enables gold probe—target analyte complex formation in a homogeneous reaction.

### Example 3

### The Use of Dextran Sulfate as a Hybridization Facilitator for Detection of Double Stranded Nucleic Targets with DNA-Modified Gold Nanoparticle Probes

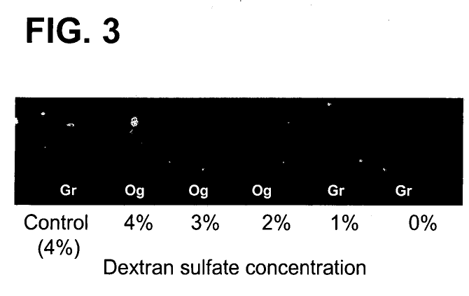

An experiment similar to that described in Example 2 was performed using a mecA gene sequence in place of the Factor V Leiden gene. A pair of 40 nm diameter gold probes (SEQ ID NO: 11 and 12) designed to bind to a mecA gene PCR amplicon (281 bp, SEQ ID NO: 14) was used in initial testing. A ˜6 nM mecA 281 base-pair PCR fragment (5 μL, fragment length and approximate concentration determined with an Agilent Bioanalyzer) was mixed with 6 μL of 40 nm diameter gold probe (1:1 ratio, 50 pM total probe), and 4 μL of hybridization buffer (buffer contains 20% formamide, 16% dextran sulfate, and 3.75 mM MgCl₂). The solutions were heated to 95° C. for 30 seconds and incubated in a water bath at 40° C. for 15 min. A 1 μL aliquot of each sample was spotted and imaged wet. As shown in FIG. 3, gold probe samples containing more than 2% dextran sulfate exhibited a color change from green to orange when hybridized for 15 minutes to a 100 fold molar excess of a 281 base-pair mecA gene PCR fragment, while no color change was observed for the same test samples that contained less than 1% dextran sulfate. A no target control containing 4% dextran sulfate also remained green, demonstrating that the color change was hybridization specific. Target solutions with less than 1% dextran sulfate remained green even after hours of hybridization demonstrating that the concentration of dextran sulfate was an important factor in this homogeneous assay format.

### Example 4

### Homogeneous SNP Identification in PCR Amplicons Using Gold Probe (30, 40, or 50) Hybridization Mediated by an Anionic Polysaccharide in Conjunction with Scatter-Based Detection

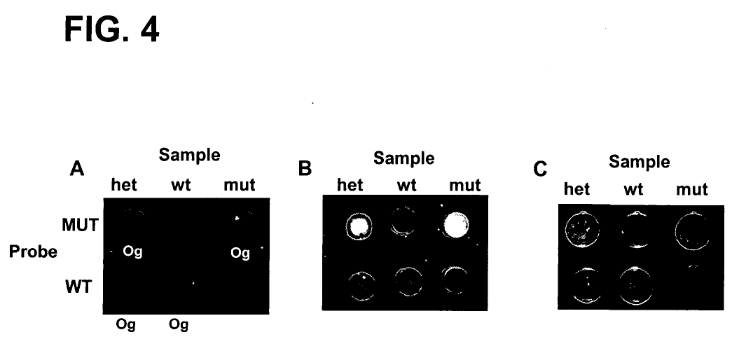

To demonstrate utility in homogeneous SNP identification, assays were designed for a single base mutation (T→A at APC nucleotide 3920) in the APC gene, which is referred to as the I1307K mutation. Wild type specific (APC 1-WT; SEQ ID NO: 1) and mutant specific (APC 1-MUT; SEQ ID NO: 2) oligonucleotide probes were designed for detection, along with a second oligonucleotide probe (APC 2; SEQ ID NO: 3) specific for a region adjacent to the SNP. The oligonucleotide probes were attached to 30, 40, and 50 nm Au probes for testing. For 30 nm Au probes, 5 ul of 2 nM wild type probe (APC-1 WT) or mutant probe (APC 1-MUT), and 5 ul of 2 nM APC2 probe, were mixed with 5 ul of wild type (SEQ ID NO: 8), mutant (SEQ ID NO: 9), or heterozygous APC gene PCR amplicons. To each of these samples, 2 uL of 25% dextran sulfate and 0.5 uL of 25 mM MgCl₂ were added. The samples were heat denatured for 30 seconds at 95° C. followed by incubation at room temperature for 10 minutes. A 1 uL aliquot of each sample was spotted onto a poly-L-lysine slide and imaged, FIG. 4. Three different illumination/detection methodologies were evaluated. In the first detection method, the plane of the glass slide is illuminated with white light generating an evanescent wave at the sample surface³⁸, and an image is captured with a color CMOS detector, FIG. 4A. In the second detection method, the plane of the glass slide is illuminated with white light, and an image of the slide is captured using a monochrome CMOS detector, FIG. 4B. In the third detection method, the plane of the glass slide is illuminated with a red LED, and an image of the slide is captured using a monochrome CMOS detector, FIG. 4C. In addition, another approach not tested is to illuminate with white light and use a specific wavelength filter to select for specific wavelengths characteristic to the gold probe complexes to enhance signal differentiations although this has not been tested to date. The three genotypes are differentiable based on the intensity of light scattered from the wild type and mutant probe reactions using all three different imaging methods. For the color CMOS imaging, the genotypes are also differentiable on the basis of color of scattered light, where the matched target/probe solutions exhibit intense orange scatter, and the mismatched target/probe solutions exhibit little to no scatter.

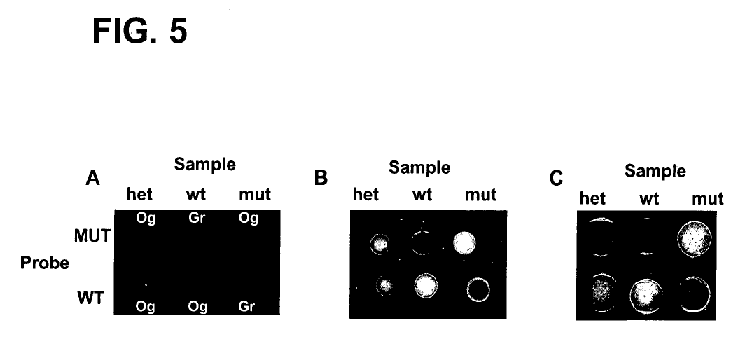

This experiment was repeated for 40 nm diameter gold particle probes using the same set of sequences, assay conditions, and protocol with the exception that the initial probe concentration was 1 nM rather than 2 nM. The results of this set of experiments are shown in FIG. 5. Again, the three genotypes are differentiable based on the intensity of light scattered from the wild type and mutant probe reactions using each imaging method. For the color CMOS imaging, the genotypes are also differentiable on the basis of color of light scattered (orange for a positive reaction, green for a negative reaction) from the wild type and mutant probe reactions, FIG. 5A. A noteworthy difference is the increase in green scatter from the 40 nm gold probe samples when compared to the 30 nm gold probe samples, FIGS. 4A and 5A. Red LED illumination minimizes this background scatter by selectively illuminating the gold probe complexes, FIG. 5C.

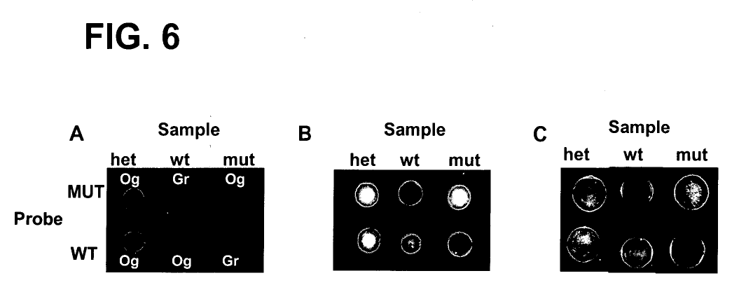

This experiment was repeated for 50 nm diameter gold particle probes using the same set of sequences, assay conditions, and protocol, with the exception that the initial probe concentration was 500 pM rather than 2 nM. The results of this set of experiments are shown in FIG. 6. The three genotypes are differentiable based on the intensity of light scattered from the wild type and mutant probe reactions using each imaging method. For the color CMOS imaging, the genotypes are also differentiable on the basis of color of light scattered (orange for a positive reaction, green for a negative reaction) from the wild type and mutant probe reactions, FIG. 6A. There is an even larger increase in green scatter from the uncomplexed 50 nm gold probe samples due to a further increase in probe size. This background scatter is minimized by using red LED illumination.

### Example 5

### Detection Sensitivity Using Anionic Polysaccharides and Scatter-Based Imaging

The principle challenge of obtaining high sensitivity bioanalyte detection in homogeneous reactions involving gold nanoparticle probes is the ability to bind probes efficiently at low (<nM) probe concentrations. Low probe concentrations are required because in a homogeneous reaction format, any probe that is not bound to target contributes to the background signal. This presents a major kinetic issue for large gold nanoparticles (>30 nm diameter) which diffuse very slowly. In addition, previous studies used probe concentrations that were equivalent or lower than the target concentration to produce a detectable color change. As a result, there have been no studies that demonstrate homogeneous binding and subsequent color changes produced with a substantial excess (>10:1) of probe over target.

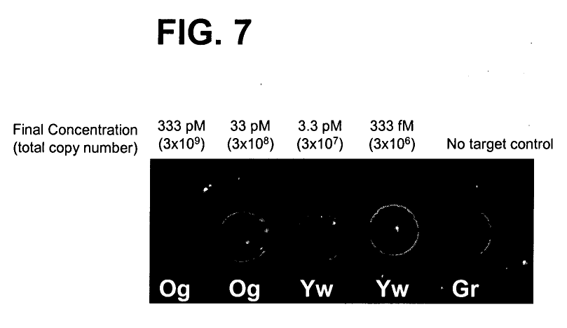

Using dextran sulfate to promote gold probe—target analyte complex formation in combination with evanescent induced light scatter detection, we tested assay sensitivity in a titration experiment using a synthetic mecA gene target, FIG. 7. Oligonucleotide probes designed to bind to adjacent regions of the mecA gene were attached to 50 nm diameter gold particles (SEQ ID NO: 11 and 12). The sequences are as follows:

Probe MecA 1:

(SEQ ID NO:11)

gold-S′-5′-[a₁₅-PEG-atggcatgagtaacgaagaata-3′]ₙ
Probe MecA 2:

(SEQ ID NO:12)

gold-S′-5′-[a₁₅-PEG-ttccagattacaacttcacca-3′]ₙ
Target MecA 3:

(SEQ ID NO:13)

5′ tgg tga agt tgt aat ctg gaa ctt gtt gag cag agg
ttc ttt ttt atc ttg ggt taa ttt att ata ttc ttc
gtt act cat gcc at 3′

Various concentrations of target (5 uL) (SEQ ID NO: 13) were mixed with 3 uL of each probe at 20 pM (SEQ ID NO: 11 and 12), and hybridization buffer consisting of 0.6 uL of 25 mM MgCl₂, 2.6 uL of 25% dextran sulfate, and 0.8 uL of formamide. A solution containing no target was used as a negative control. The solutions were heated at 95° C. for 30 seconds, followed by incubation at room temperature for two hours. A 1 uL aliquot of the solution was pipetted onto a poly-L-lysine slide, and the slide was placed in a dessicator until the samples were dried onto the slide. This procedure concentrated the samples onto the glass slide intensifying the amount of scattered light from each sample. The slide was illuminated with white light in the plane of the glass slide, and imaged visually or using a color CMOS, FIG. 7. A change in scatter color from green (the negative control) to yellow was observed for the lowest target concentration tested (3×10⁶ total copies in 15 uL, 333 femtomolar concentration). Above 3×10⁸ total copies, a change in scatter color from green to orange was observed. Thus, no more than 2×10⁵ target molecules (333 zmol) were required under these assay conditions for visual detection of the color change in a 1 uL spot. This sensitivity was roughly 4 orders of magnitude higher than the previously reported 10 fmol detection limit (3×10⁹ copies/μL in a 2 μL reaction) achieved by visual analysis of reflected light from aliquots spotted onto a thin layer chromatography plate⁹.

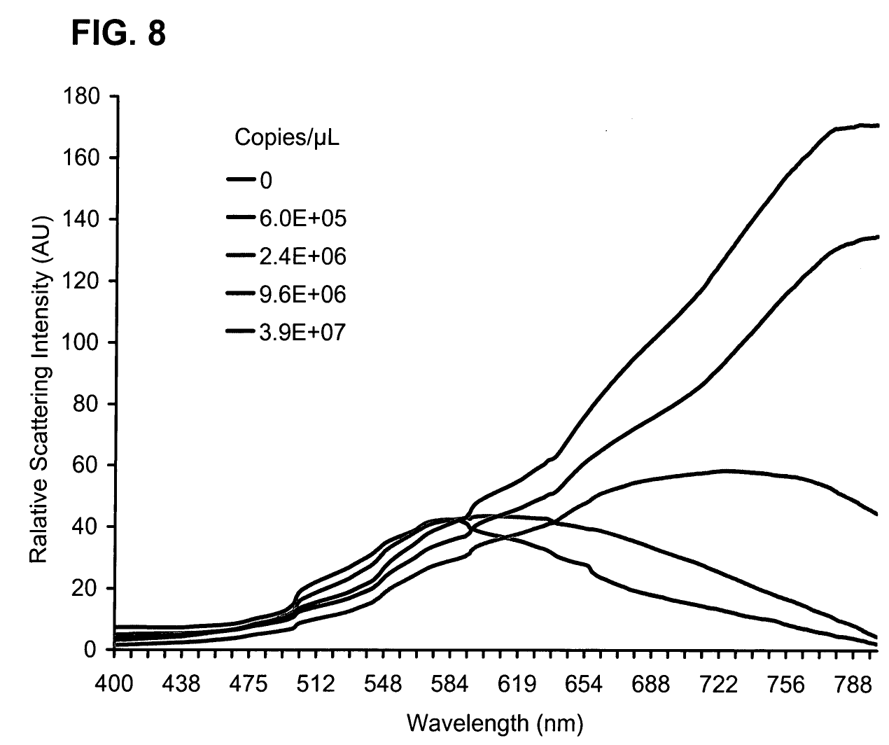

This substantial increase in sensitivity may be attributed to three main factors. First, monitoring scattered light enabled detection of significantly lower nanoparticle concentrations when compared to detection of absorbed or reflected light^{36,39,40}. The samples from this experiment could not be detected visually when a 1 μl aliquot was spotted onto a reverse phase plate because the total probe concentration was only 8 picomolar. Second, assuming every target was complexed with two particles, there was an approximate 24 fold excess of non-aggregated particles at the lowest target concentration when a color change was still observable. Thus, target hybridization can be driven with an excess of probe, and lower concentrations of target can be detected with nanoparticle concentrations visually detectable by scatter. The 50 nm diameter particles were expected to promote a larger colorimetric shift than the 15 nm diameter particles due to increased gold volume¹⁶, but this surprising result also indicated that the probe-target complexes must exhibit a large increase in scatter intensity compared to individual probes (also see example 6, FIG. 8). Third, the use of dextran sulfate enhanced hybridization kinetics⁴¹, permitting rapid probe-target hybridization even at low (picomolar) concentrations of probe (also see Example 2).

### Example 6

### Gold Nanoparticle Probe Complexes Increase Scatter Intensity

The relative scattering intensity of probe/target complexes can be monitored using a diode array detector, FIG. 8. Five microliter target samples (SEQ ID NO: 13) were mixed with 6 μL of 50 nm diameter gold probes (SEQ ID NO: 12 and 15) (1:1 ratio, 20 pM total probe), and 4 μL of hybridization buffer (containing 20% formamide, 16% dextran sulfate, and 3.75 mM MgCl₂). A solution containing hybridization buffer and both probes without target was used as a negative control. The solutions were heated at 95° C. for 30 seconds, followed by incubation at room temperature for two hours. A 1 uL aliquot of the solution was spotted, and the slide was placed in a dessicator until the samples were dried prior to imaging. The resulting samples were then spotted and dried onto poly-L-lysine slides and placed in an evanescent illuminator. The spectra were collected using a USB 2000 photodiode spectrometer from Ocean Optics, Inc. configured for operation in the 350-1000 nm range, FIG. 8. To establish spectral correction factors accounting for nuances in the spectrophotometer and the light source, all samples were normalized to a filtered solution of Ludox TM-50. As the target concentration increases, the scattering intensity increases in the wavelength region of 600-800 nm. Given that the overall probe concentration was the same for each experiment, it was concluded that the probe-target complex formation substantially enhanced scatter intensity. Consequently, probe-target complexes can be detected in a background of uncomplexed probes as described in Example 5.

### Example 7

### Formation of Gold Nanoparticle Probe Complexes

To demonstrate that the scatter-based color and intensity changes were due to the formation of gold probe complexes and not single particles binding to nucleic acid targets, a control experiment was performed wherein a mecA 281 base-pair PCR amplicon (SEQ ID NO: 14) was hybridized to a single complementary 50 nm diameter gold probe sequence in solution (MecA 1 SEQ ID NO: 11 or MecA 2 SEQ ID NO: 12), or to two gold probes complementary to the target in the same solution (MecA 1 and 2 together, SEQ ID NO's: 11 and 12).

(SEQ ID NO:14)

Target MecA 4
5′ATCCACCCTCAAACAGGTGAATTATTAGCACTTGTAAGCACACCTTCA
TATGACGTCTATCCATTTATGTATGGCATGAGTAACGAAGAATATAATAA
ATTAACCGAAGATAAAAAAGAACCTCTGCTCAACAAGTTCCAGATTACAA
CTTCACCAGGTTCAACTCAAAAAATATTAACAGCAATGATTGGGTTAAAT
AACAAAACATTAGACGATAAAACAAGTTATAAATCGATGGTAAAGGTTGG
CAAAAAGATAAATCTTGGGGTGGTTACAACGT 3′

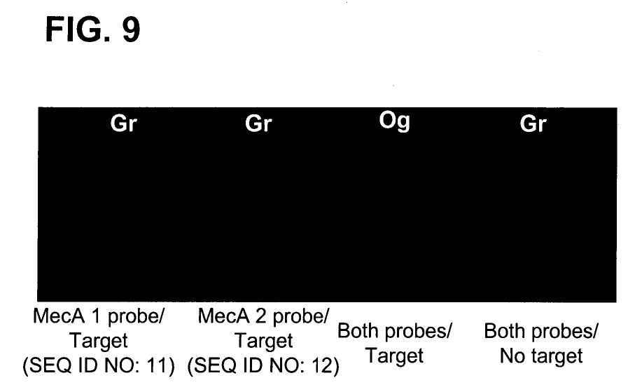

The reaction conditions were 80 pM total probe, 1 mM MgCl₂, 4.33% dextran sulfate, 5.33% formamide, and 13 nM of mecA 281 bp PCR product (SEQ ID NO: 14). A solution with both probes in the absence of target was used as a negative hybridization control. The solutions were denatured at 95° C. for thirty seconds, hybridized for two hours at room temperature, and subsequently a 1 ul aliquot was spotted onto a poly lysine coated glass slide and imaged, FIG. 9. The light scattered by the solution with both probes was an intense orange color, while the solutions containing a single probe or no target exhibit much weaker green scatter. This experiment demonstrates that both probes are required to see a scatter change.

### Example 8

### MecA Gene Detection From Unamplified Genomic DNA Using 50 nm Gold Probes

To test the feasibility of directly detecting DNA sequences without using enzyme-based target or signal amplification procedures, an experiment was conducted for detection of the mecA gene in clinical samples of methicillin resistant S. aureus (MRSA)⁴². The mecA gene confers resistance to the antibiotic methicillin. In addition to high sensitivity, this also requires high selectivity since the probes must hybridize specifically to the target sequence in the presence of highly complex DNA. 50 nm diameter gold probes (SEQ ID NO: 11 and 12) that hybridize to adjacent regions of the mecA gene were used for detection.

Probe MecA 1:

(SEQ ID NO:11)

gold-S′-5′-[a₁₅-PEG-atggcatgagtaacgaagaata-3′]ₙ
Probe MecA 2:

(SEQ ID NO:12)

gold-S′-5′-[a₁₅-PEG-ttccagattacaacttcacca-3′]ₙ

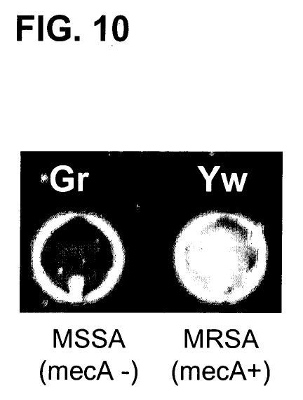

Purified genomic DNA samples for both a methicillin positive bacterium (MRSA) and methicillin negative bacterium (MSSA) were ordered from ATCC (Item Nos. 700699D and 35556D, respectively) and resuspended in 20 mM Tris to a final concentration of 1 μg/μL. The samples were sonicated on ice using 3 consecutive 10 second, 3-watt pulses and then diluted with Tris buffer to a final concentration of 100 ng/μL. A 1 μL aliquot of each genomic DNA solution was then combined with 6 μL of a probe solution containing 10 pM of each probe (SEQ ID NO: 11 and 12). Subsequently, 4 μL of a hybridization solution containing 3.75 mM MgCl₂, 16% (V/V) dextran sulfate, and 18.75% (V/V) formamide was added to the sample. The entire mixture was heated to 95° C. for 30 seconds, followed by incubation at 37° C. for 45 minutes. One microliter of each sample was then spotted on a poly-L-lysine slide, and the slide was evanescently illuminated with white light and imaged visually or with a color CMOS sensor while the solutions remained wet, FIG. 10. Light scattered from the negative control MSSA (mecA−) genomic DNA sample appeared green while the MRSA (mecA+) appeared as a bright yellow-orange. These results demonstrated that this assay can be used to detect specific gene sequences in unamplified genomic DNA. To our knowledge, this is the first example of a homogeneous assay that can detect genomic DNA sequences without enzymatic intervention.

### Example 9

### MecA Gene Detection from Unamplified Genomic DNA Samples Using 50 nm Gold Probes

The scatter-based calorimetric assay also can be used to detect specific gene sequences from unamplified genomic DNA samples after the samples are dried onto the slide. This procedure can enhance detection sensitivity by decreasing interparticle distance. This principle was demonstrated by detecting the mecA gene from purified Staphylococcus aureus genomic DNA samples that were fragmented by sonication. In this example, two 50 nm gold probes (SEQ ID NO: 11 and 12, respectively) that hybridize to adjacent regions of the mecA gene were used for detection.

Probe MecA 1:

(SEQ ID NO:11)

gold-S′-5′-[a₁₅-PEG-atggcatgagtaacgaagaata-3′]ₙ
Probe MecA 2:

(SEQ ID NO:12)

gold-S′-5′-[a₁₅-PEG-ttccagattacaacttcacca-3′]ₙ

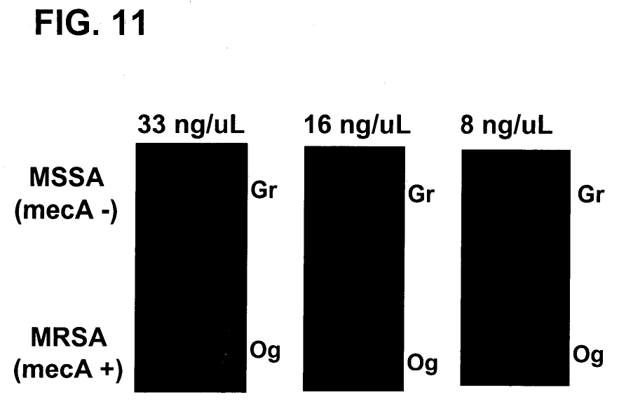

Purified Staphylococcus aureus genomic DNA samples for both a methicillin resistant bacterium (MRSA) and methicillin sensitive bacterium (MSSA) were ordered from ATCC (Item Nos. 700699D and 35556D, respectively) and resuspended in 20 mM Tris to a final concentration of 1 μg/μL. The samples were sonicated on ice using three, consecutive 10-second, 3-watt pulses, and then diluted with Tris buffer to a final concentration of 100 ng/μL, 50 ng/μL, or 25 ng/μL. For the assay, a 1 μL aliquot of each genomic DNA sample was combined with 1.2 μL of a probe solution containing 12.5 pM of each probe (SEQ ID NO: 11 and 12). Subsequently, 0.6 μL of a hybridization solution consisting of 3.75 mM MgCl₂, 16% (V/V) dextran sulfate, and 18.75% (V/V) formamide was added to the sample. The entire mixture was heated to 95° C. for 30 seconds, followed by incubation at 37° C. for 40 minutes. One microliter of each sample was then spotted on a poly-L-lysine slide at 43° C. and allowed to dry for 10 minutes. The slide was then evanescently illuminated with white light, and the color of scattered light from each spotted sample was imaged visually or with a color CMOS sensor, FIG. 11. The methicillin resistant S. aureus (MRSA) samples were detectable by either method at concentrations as dilute as 8 ng/μL based on a change in scatter color from green to orange. The light scattered by the methicillin sensitive S. aureus (MSSA) negative control samples appeared green in color indicating no significant non-specific binding with background genomic DNA under these detection conditions. This experiment demonstrates that specific gene sequences can be detected from unamplified genomic DNA samples after the samples are spotted and dried onto the slide.

### Example 10

### Signal Quantitation and Reproducibility of MecA Gene Detection from Unamplified Genomic DNA Samples Using Homogeneous Detection with Gold Nanoparticle Probes

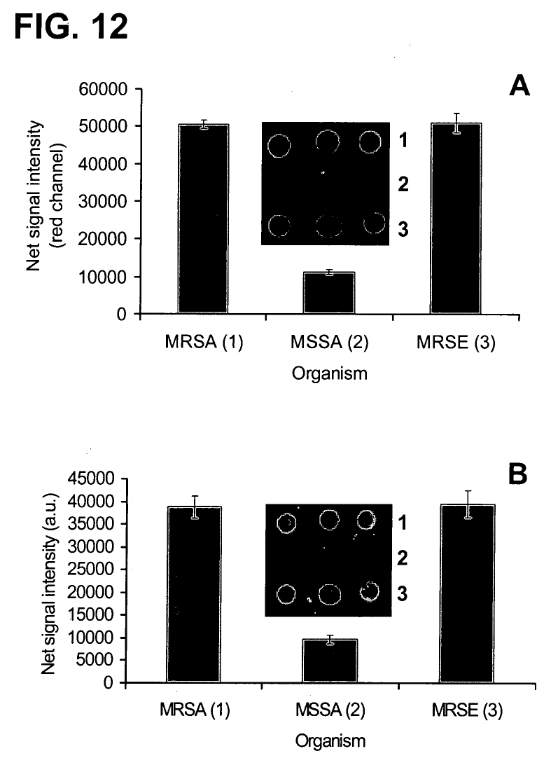

In addition to visual analysis, the colorimetric signals generated by this assay can be quantified by analyzing signal intensity in the red channel of an RGB sensor such as a color CCD. For two probe detection, a 100 ng/μL solution of MRSA, MSSA, and MRSE (2 μL) was combined with 2.4 ul of 40 nm diameter gold probes (sequence ID NO's: 11 and 12 at a 1:1 ratio, 25 pM total probe), and 1.6 μL of hybridization buffer (consisting of 3.75 mM MgCl₂, 16% (V/V) dextran sulfate, and 18.75% (V/V) formamide). The entire mixture was heated to 95° C. for 1.5 minutes, followed by incubation at 37° C. for one hour. One μL of each sample was spotted onto a slide at 43° C. and allowed to dry for 10 minutes. The evanescent induced scatter from the glass slide was imaged with a color CMOS sensor. The net signal intensity in the red channel of the color CMOS sensor was quantified for each spot (three replicates of each genomic DNA sample tested) using Genepix software from Axon instruments, FIG. 12A. Signal quantitation also was achieved using the Verigene™ ID detection system designed at Nanosphere (Northbrook, Ill.), which illuminated the glass slide with a red LED (λₑₓ=630 nm central wavelength) and captured an image of the entire glass slide using a monochrome photosensor, FIG. 12B. The net signal intensity from each spot was quantified using Genepix software.

Analysis of methicillin resistant and sensitive Staphylococcus genomic DNA samples demonstrates that the visually observed colorimetric changes for methicillin resistant samples are quantifiable by both imaging methods with signals that are 3-5 fold greater than the methicillin sensitive samples, and >3 standard deviations above the negative control methicillin sensitive samples, FIG. 12. In addition, the signals generated by this assay are reproducible.

### Example 11

### MecA Gene Detection from Unamplified Genomic DNA Samples Using More than Two 50 nm Gold Probes

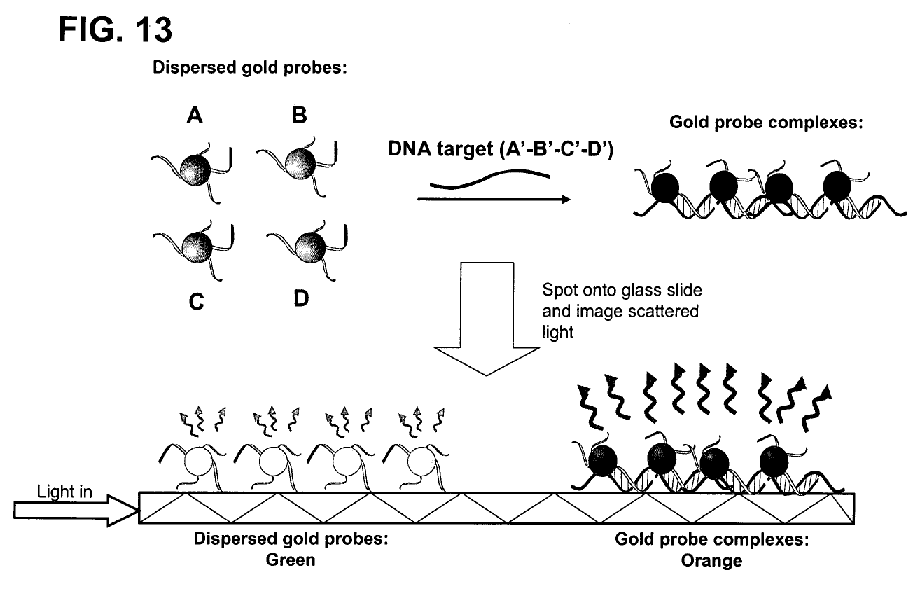

Since the colorimetric shift is dependent on the number of particles within the aggregate structure and the distance between the particles¹⁰, we reasoned that increasing the number of gold probes per target may provide higher sensitivity by enhancing the plasmon band red-shift. Two additional mecA gene probes (SEQ ID NOs: 15 and 16) were designed to bind in close proximity to the existing probes (SEQ ID NOs: 11 and 12) to test this hypothesis, FIG. 13. 50 nm diameter gold probes were prepared with each sequence for testing.

Probe MecA 1:

(SEQ ID NO:11)

gold-S′-5′-[a₁₅-PEG-atggcatgagtaacgaagaata-3′]ₙ
Probe MecA 2:

(SEQ ID NO:12)

gold-S′-5′-[a₁₅-PEG-ttccagattacaacttcacca-3′]ₙ
Probe mecA 3:

(SEQ ID NO:15)

gold-S′-5′-[a₁₅-PEG-aaagaacctctgctcaacaag-3′]ₙ
Probe mecA 4:

(SEQ ID NO:16)

gold-S′-5′-[a₁₅-PEG-gcacttgtaagcacaccttcat-3′]ₙ

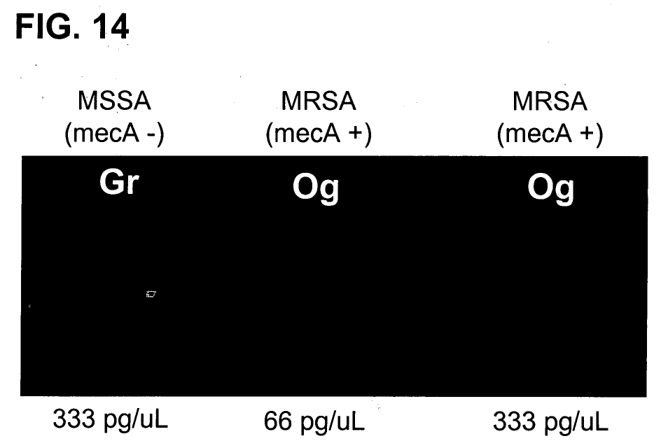

Methicillin resistant (mecA+) and sensitive (mecA−) Staphylococcus aureus genomic DNA samples isolated from cultured bacterial cells were purchased from ATCC (Item Nos. 700699D and 35556D, respectively) and resuspended in 20 mM Tris to a final concentration of 1 μg/μL. The samples were sonicated on ice using three consecutive 10 second, 3-watt pulses and then diluted with Tris buffer to a final concentration of 1 ng/μL or 200 pg/μL. For the assay, five microliters of each genomic DNA sample was combined with 6 μL of a probe solution containing 5 pM of each gold probe (SEQ ID NO: 11, 12, 15, and 16) in 20 mM Tris buffer. Subsequently, 4 μL of a hybridization solution consisting of 3.75 mM MgCl₂, 16% (V/V) dextran sulfate, and 18.75% (V/V) formamide was added to the sample. The entire mixture was heated to 95° C. for 30 seconds, followed by incubation at 37° C. After 2 hours, one microliter of each sample was spotted on a poly-L-lysine slide at 43° C. and allowed to dry for 10 minutes. The slide was then evanescently illuminated with white light, and the color of scattered light from each spotted sample was imaged visually or with a color CMOS sensor, FIG. 14. The light scattered by positively detected samples will appear yellow to orange in color, whereas negative samples will scatter light that appears green in color.

The methicillin resistant S. aureus (mecA+) samples were detectable at concentrations as low as 66 pg/uL based on a change in scatter color from green to orange. The light scattered by the methicillin sensitive S. aureus (mecA−) negative control sample remained green in color indicating minimal non-specific binding to the background genomic DNA sequences present in the sample. 66 picograms of methicillin resistant S. aureus genomic DNA target was equivalent to approximately 20,000 copies (33 zmol) in the 1 μL volume analyzed on the glass slide, which represented an order of magnitude increase in sensitivity when compared to the same analysis with only two DNA-GNP₅₀ probes. Equally important was the increase in the detectable probe/target ratio (˜230 fold), which suggested that a four probe complex enhanced both the colorimetric shift and scatter intensity when compared to a two probe complex.

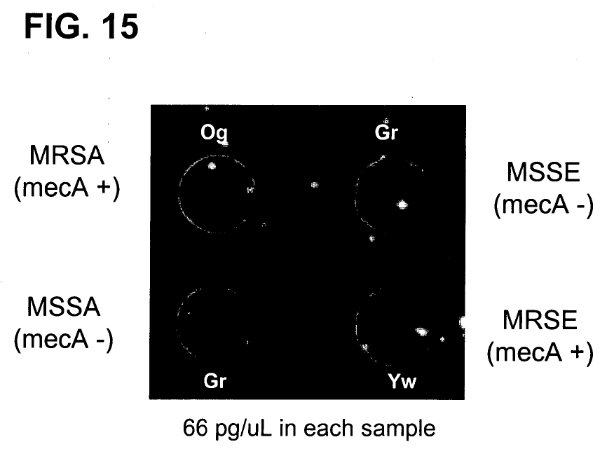

Other methicillin resistant (mecA+) and sensitive (mecA−) genomic DNA samples isolated from cultured S. epidermidis bacterial cells (MRSE: Item No. 12228D and MSSE: Item No. 35984D obtained from ATCC) were tested using similar DNA preparation, assay, and imaging conditions, FIG. 15. The methicillin resistant S. epidermidis (mecA+) sample exhibited a change in scatter color from green to yellow while the light scattered by the methicillin sensitive S. epidermidis (mecA−) negative control sample remained green in color. The ability to effectively detect the mecA gene in unamplified genomic DNA samples isolated from multiple Staphylococcus subspecies further demonstrates the specificity of the scatter-based colorimetric detection assay.

### Example 12

### Preparation of Antibody-Coated Gold Probes

Polyclonal anti-IgE antibodies are purchased from Chemicon International Inc. (Temecula, Calif.). Gold nanoparticles 40-60 nm in diameter are purchased from Ted Pella, Inc (Redding, Calif.). The anti-IgE antibodies are attached to gold nanoparticles via direct binding of the antibody to the gold particle (referred to as passive adsorption). The procedure was adapted from an existing protocol developed by British Biocell International. Briefly, the 40-60 nm diameter gold particles are adjusted to a pH between 9-10 using sodium carbonate buffer. 3 μg/mL of the antibody is added to the gold nanoparticle and incubated at room temperature for 1.0 hour. Next, the probes are filtered through 0.2 um diameter cellulose acetate, and then BSA is added to stabilize the particles. The antibody-gold nanoparticle conjugates are isolated by centrifugation at 2100 G for 25 minutes. The supernatant is removed following centrifugation, and the particles are redispersed in buffer (20 mM Tris buffer (pH 8.5), 0.1% BSA and 0.01% azide). The final nanoparticle concentration is measured by UV-visible absorbance at 520 nm using an estimated extinction coefficient of ε₅₂₀=1.5×10¹⁰ M⁻¹cm⁻¹ for the 50 nm diameter gold particles and ε₅₂₀=2.8×10¹⁰ M⁻¹cm⁻¹ for the 60 nm diameter gold particles.

It should be noted that the probes are brought to 0.2 M NaCl, and the amount of probe aggregation is measured by analyzing the scatter color on a waveguide substrate prior to the BSA step. This ensures that the probes are stable and sufficient antibody is attached during the preparation process.

### Example 13

### IgE Detection with Anti-IgE Antibody Coated Gold Probes

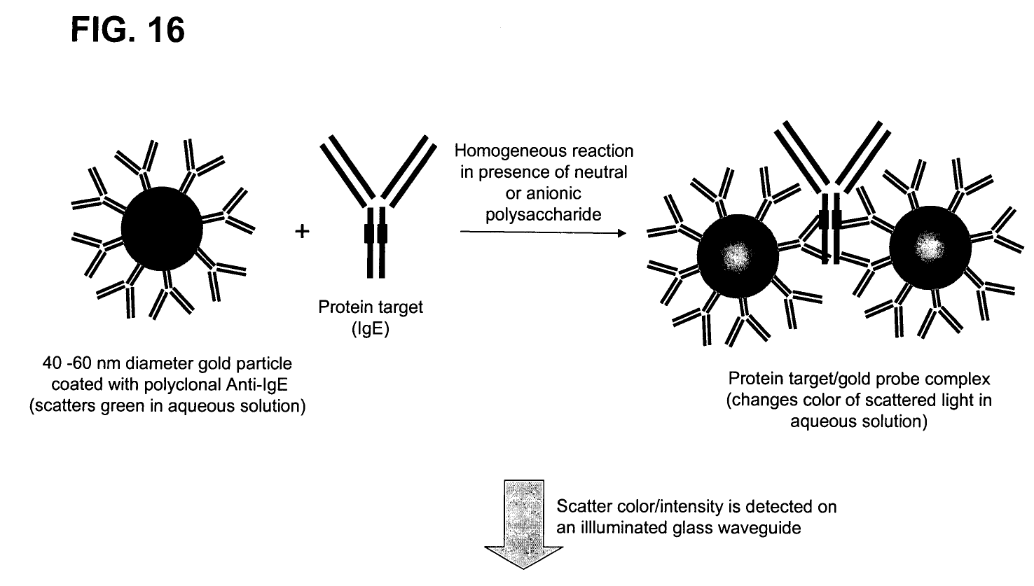

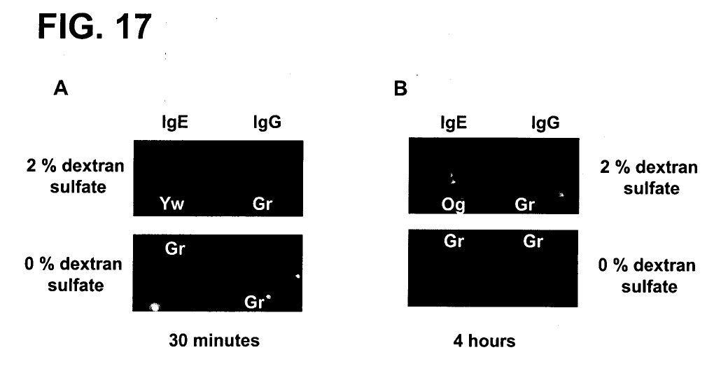

Evanescent illumination and scatter-based detection of gold probe complexes formed through antibody-antigen interactions was initially tested using 50 and 60 nm diameter gold probes coated with anti-IgE polyclonal antibody, FIG. 16. Binding of the anti-IgE coated gold probes to IgE target was tested with and without dextran sulfate. For testing, ten microliter samples containing IgE target (2 ul of 250 ng/mL) or an IgG negative control (2 ul of 250 ng/mL), 50 nm diameter anti-IgE gold probe (3 ul of 250 pM), and buffer (5 ul of 2×PBS, 2 mM MgCl₂ or 2×PBS, 2 mM MgCl₂, 4% dextran sulfate) were prepared. One microliter of each sample was spotted onto a poly-L-lysine glass slide after 30 minutes, and after 4 hours of incubation. The glass slide was illuminated with white light in the plane of the slide, and the color of scatter light from each sample was observed visually or captured with a color CMOS detector, FIG. 17. After 30 minutes, light scattered from the IgE target sample containing dextran sulfate was a visually detectable yellow, while light scattered from the IgG negative control was a visually detectable green. By contrast, the light scattered from both the IgE target and IgG negative control samples without dextran sulfate was a visually detectable green. After 4 hours, light scattered by the IgE target sample containing dextran sulfate was a visually detectable orange, while the light scattered from the the samples without dextran sulfate remained a visually detectable green. These experiments demonstrate that dextran sulfate promotes the formation of antibody gold probe-antibody target complexes which leads to high sensitivity detection by evanescent induced colorimeter scatter.

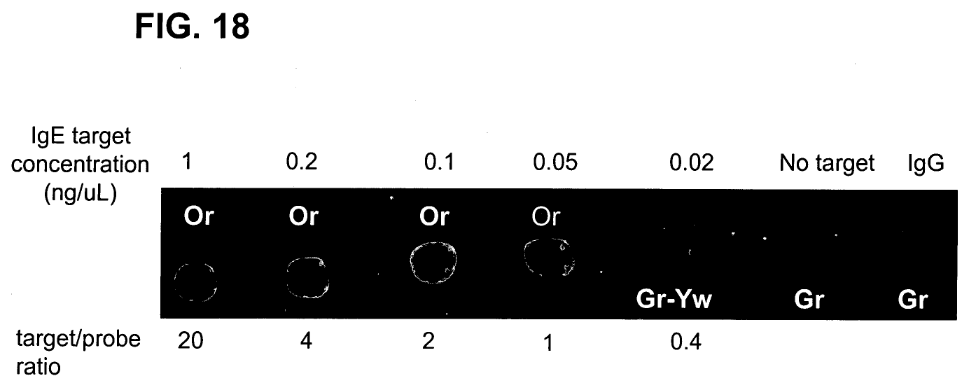

The IgE target was titrated into 40 m diameter anti-IgE gold probes to assess assay sensitivity. For the assay, 5 uL of 40 nm anti-IgE gold probe (500 pM) is added to 2 uL of IgE target (5, 1, 0.5, 0.25, or 0.1 ng/μL), IgG negative control, or water as a no target control, and 3 uL of incubation buffer consisting of 6.5% dextran sulfate, 5 mM MgCl₂, and 3.5×PBS. Each sample is incubated at room temperature for 1 hour. A 1 ul droplet of each sample is subsequently transferred onto a glass slide illuminated with white light, and the scattered light from each deposited sample is captured visually or with a CMOS camera, FIG. 18. The color of light scattered by the IgE target samples at >0.05 ng/uL was orange, while the color of light scattered by the IgG and no target control samples was a visually differentiable green. The red-shift in scatter color for the IgE target samples is attributed to the formation gold probe complexes that result from binding of the IgE target. These optical changes may be visually detected or imaged and quantitated as described in Examples 4 and 10.

### Example 14

### Imaging Calorimetric Changes Associated with Individual Gold Probe Complexes Using Evanescent Illumination and High Resolution Optics

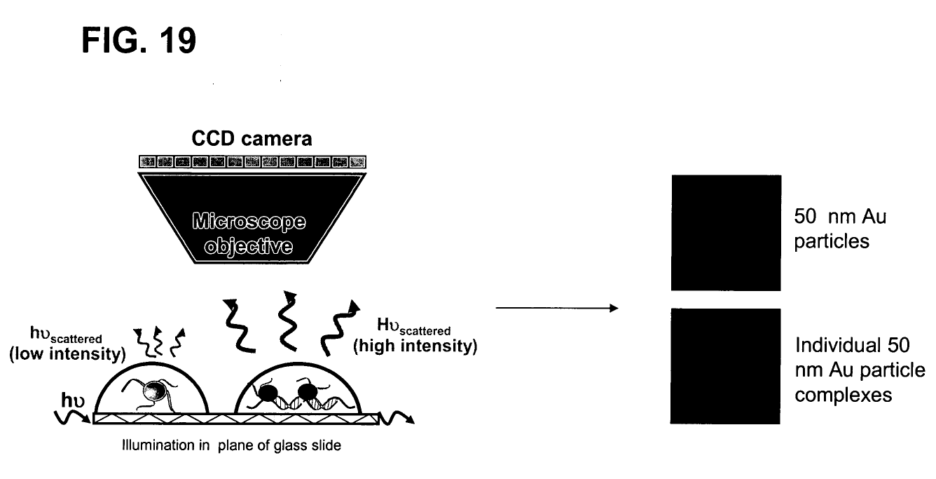

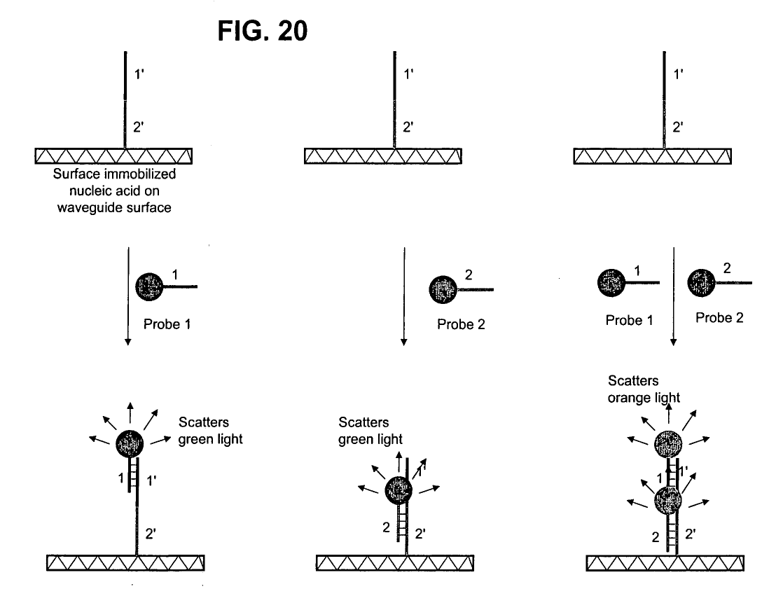

A 1 ul aliquot of 50 nm diameter gold particles or gold probe complexes formed through DNA hybridization were spotted onto a glass slide, and the scatter from the solutions was imaged in the solution state via an evanescent wave generated with white light illumination in the plane of the glass slide, FIG. 19. The scattered light was collected through a 10-100× objective onto a color CCD camera. White light illumination in the plane of the glass slide and collection through a 10-100× objective onto a color CCD camera provides high resolution images of individual particle scatter in the solution state which is green in color (the particles appear larger since the optical resolution is diffraction limited). More importantly, the individual gold probe complexes are also detectable in solution, with a concomitant change is the color of scattered light ranging from yellow to red, FIG. 19. This demonstrates that colorimetric changes associated with the formation of individual gold probe complexes is feasible using high resolution optics.

### Example 15

### Preparation of Nanoparticle-Oligonucleotide Conjugate Probes

In this Example, a representative nanoparticle-oligonucleotide conjugate detection probe was prepared for use in the detection of surface immobilized nucleic acid targets.

(a) 50 nm Diameter Gold Nanoparticles

Solutions of 50 nm diameter gold particles were purchased from Ted Pella, Inc. for the described experiments.

(b) Synthesis of Steroid Disulfide Modified Oligonucleotides (SDO)

Oligonucleotides were synthesized on a 1 micromole scale using a Applied Biosystems Expedite 8909 DNA synthesizer in single column mode using phosphoramidite chemistry. Eckstein, F. (ed.) Oligonucleotides and Analogues: A Practical Approach (IRL Press, Oxford, 1991). All synthesis reagents were purchased from Glen Research or Applied Biosystems. Average coupling efficiency varied from 98 to 99.8%, and the final dimethoxytrityl (DMT) protecting group was removed from the oligonucleotides so that the steroid disulfide phosphoramidite could be coupled.

To facilitate hybridization of the probe sequence with the target, a deoxyadenosine oligonucleotide-polyethylene glycol (dA₁₀-PEG) was included on the 5′ end in the probe sequence as a spacer.

To generate 5′-terminal steroid-cyclic disulfide oligonucleotide derivatives (see Letsinger et al., 2000, Bioconjugate Chem. 11:289-291 and PCT/US01/01190 (Nanosphere, Inc.), the disclosure of which is incorporated by reference in its entirety), the final coupling reaction was carried out with a cyclic dithiane linked epiandrosterone phosphoramidite on Applied Biosystems automated Expedite 8909 synthesizer, a reagent that prepared using trans 1,2-dithiane-4,5-diol, epiandrosterone and p-toluenesulphonic acid (PTSA) in presence of toluene. The phosphoramidite reagent may be prepared as follows: a solution of epiandrosterone (0.5 g), trans 1,2-dithiane-4,5-diol (0.28 g), and p-toluenesulfonic acid (15 mg) in toluene (30 mL) was refluxed for 7 h under conditions for removal of water (Dean Stark apparatus); then the toluene was removed under reduced pressure and the reside taken up in ethyl acetate. This solution was washed with 5% NaHCO₃, dried over sodium sulfate, and concentrated to a syrupy reside, which on standing overnight in pentane/ether afforded a steroid-dithioketal compound as a white solid (400 mg); Rf (TLC, silica plate, ether as eluent) 0.5; for comparison, Rf values for epiandrosterone and 1,2-dithiane-4,5-diol obtained under the same conditions are 0.4, and 0.3, respectively. The compound was purified by column chromatography. Subsequently, recrystallization from pentane/ether afforded a white powder, mp 110-112° C.; ¹H NMR, δ 3.6 (1H, C³OH), 3.54-3.39 (2H, m 2OCH of the dithiane ring), 3.2-3.0 (4H, m 2CH₂S), 2.1-0.7 (29H, m steroid H); mass spectrum (ES⁺) calcd for C₂₃H₃₆O₃S₂ (M+H) 425.2179, found 425.2151. Anal. (C₂₃H₃₇O₃S₂) S: calcd, 15.12; found, 15.26. To prepare the steroid-disulfide ketal phosphoramidite derivative, the steroid-dithioketal (100 mg) was dissolved in THF (3 mL) and cooled in a dry ice alcohol bath. N,N-diisopropylethylamine (80 μL) and β-cyanoethyl chlorodiisopropylphosphoramidite (80 μL) were added successively; then the mixture was warmed to room temperature, stirred for 2 h, mixed with ethyl acetate (100 mL), washed with 5% aq. NaHCO₃ and with water, dried over sodium sulfate, and concentrated to dryness. The residue was taken up in anhydrous acetonitrile and then dried under vacuum; yield 100 mg; ³¹P NMR 146.02. The epiandrosterone-disulfide linked oligonucleotides were synthesized on Applied Biosystems Expedite 8909 gene synthesizer without final DMT removal. After completion, epiandrosterone-disulfide linked oligonucleotides were deprotected from the support under aqueous ammonia conditions and purified on HPLC using reverse phase column.

Reverse phase HPLC was performed with a Dionex DX500 system equipped with a Hewlett Packard ODS hypersil column (4.6×200 mm, 5 mm particle size) using 0.03 M Et₃NH⁺ OAc⁻ buffer (TEAA), pH 7, with a 1 mL/min. gradient of 95% CH₃CN/5% TEAA. The flow rate was 1 mL/min. with UV detection at 260 nm. Preparative HPLC was used to purify the DMT-protected unmodified oligonucleotides. After collection and evaporation of the buffer, the DMT was cleaved from the oligonucleotides by treatment with 80% acetic acid for 30 min. at room temperature. The solution was then evaporated to near dryness, water was added, and the cleaved DMT was extracted from the aqueous oligonucleotide solution using ethyl acetate. The amount of oligonucleotide was determined by absorbance at 260 nm, and final purity assessed by reverse phase HPLC.

(c) Attachment of SDOs to 50 nm Diameter Gold Particles

Solutions of 50 nm diameter gold particle were used as delivered from Ted Pella, Inc. The gold nanoparticle probes were prepared by loading the gold particles with steroid disulfide modified oligonucleotides using a modification of a previously developed literature procedure¹⁵. Briefly, 8 nmol of SDO was added per 3 mL of gold nanoparticle and incubated for 15 hours at room temperature. After 24 hours, aqueous sodium dodecyl sulfate (SDS, 10% by weight) was added to the solution (final concentration: 0.01%). Then, aqueous 2 M NaCl was added to a final concentration of 0.1 M NaCl. After standing for 24 additional hours, the NaCl concentration was increased to 0.2 M. This was repeated the following day to bring the NaCl concentration of the probe solution to 0.3 M. After 24 additional hours, the SDO-gold nanoparticle conjugates were isolated with a Beckman Coulter Microfuge 18 by centrifugation (5000 rpm for 25 minutes for 30 nm, 3000 rpm for 15 minutes for 40 nm, 3000 rpm for 15 minutes for 50 nm). After centrifugation, a dark red gelatinous residue remained at the bottom of the eppendorf tube. The supernatant was removed, and the conjugates were washed (2×) with 0.1 M NaCl, 10 mM phosphate (pH 7) (original colloid volume) and redispersed in 20 mM Tris HCL (pH 7).

The following nanoparticle-oligonucleotide conjugates specific for segments of the mecA gene were prepared in this manner:

Probe 1:

(SEQ ID NO:17)

gold-[S′-5′-A₁₀-PEG-ATGGCATGAGTAACGAAGAATA 3′]ₙ
Probe 2:

(SEQ ID NO:18)

gold-[S′-5′-A₁₀-PEG-TTCCAGATTACACTTCACCA3′]ₙ

### Example 16

### Changes in Scatter Color Based on Formation of Individual Probe Complexes

The target sequences used for this study is as follows:

| | | |
|---|---|---|
| DNA Target: | 5′ TGGTAAGTTGTAATCTGGAAC | (SEQ ID NO:19) |
| | TTGTTGAGCAGAGGTTCTTTTTTA | |
| | TCTTCGGTTAATTTATTATATTCT | |
| | TCGTTACTCATGCCAT 3′ | |

The nucleic acid target (SEQ ID NO: 19) was purchased from IDT and suspended in a 50% DMSO solution at ten-fold dilutions from 275 nM to 2.75 nM. Each dilution of the nucleic acid target was spotted onto an amine modified glass surface (Corning GAPS II) using an Affymetrix pin and ring robotic microarrayer and allowed to dry in a desiccator before being stored at ambient conditions. Steroid disulfide modified oligonucleotides (SEQ ID NO: 17 and SEQ ID NO: 18) complementary to the nucleic acid target were conjugated to 50 nm diameter gold particles as described herein to produce gold probes 1 and 2. Both probes 1 and 2 (SEQ ID NO: 17 and SEQ ID NO: 18, respectively) were diluted to 100 pM with 20 mM Tris at pH 7.

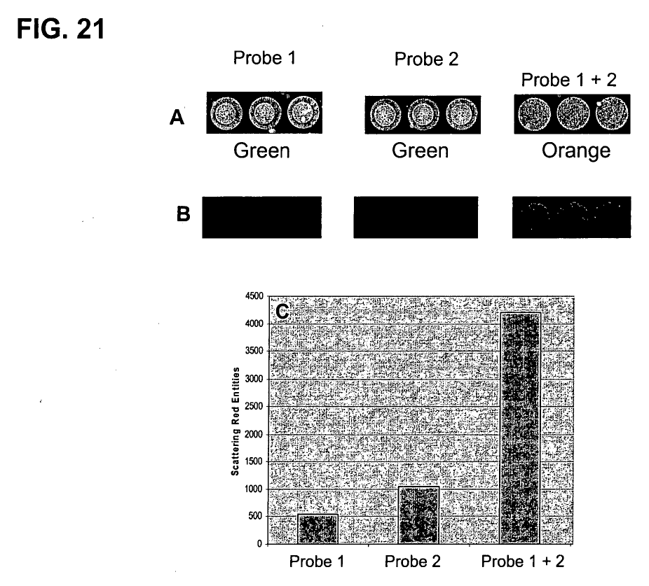

Prior to hybridization, the slides with immobilized nucleic acid target were washed briefly with water, dried, irradiated with 1000 μJ of UV light, washed with water once again and dried. Probe samples comprised of 100% Probe 1, 100% Probe 2, or a 1:1 ratio of Probe 1 and Probe 2 were prepared by combining 48 μL of 100 pM probe (24 uL of each probe for 1:1 ratio) with 40 μL of water and 32 μL of a buffer containing: 18.75% formamide, 8.13% 500 kDa dextran sulfate, and 2 mM MgCl₂. Next, 50 μL of each probe sample was pipetted into separate wells on the slide containing immobilized target and sealed. The slide was inverted and incubated at 41° C. for 2 hrs. The slides were washed in 1 mM MgCl₂ for 10 sec at room temperature (22-24 C) and allowed to dry. Planar illumination of the slide with white light generated an evanescent field at the slide surface. The color of scattered light was captured using a Zeiss Axioskop MAT microscope equipped with a Zeiss AxioCam HRc color camera at 2.5× magnification. The samples containing only probe 1 or only probe 2 exhibited a green color demonstrating that each probe binds to the nucleic acid target immobilized on the slide surface (FIG. 21). In addition, a green scatter color indicates that the hybridized 50 nm gold nanoparticles are separated by a large enough distance to prevent a color change. A change in scatter color from green to orange was observed for the sample containing a 1:1 ratio of probes 1 and 2, which indicates that both probes bind to the nucleic acid target. At 10× magnification, individual scattering entities were clearly visible; by averaging the number of scattering entities of each of the 3 repeat spots in the red channel, a substantially larger number of pixels was observed when both probes bind to the same target which results in the frequency change of the nanoparticle plasmon band.

This data demonstrated that a gold particle complex comprised of two or more particles can be detected and distinguished on the basis of color from individual gold particles hybridized or non-specifically bound to a glass slide.

### Example 17

### Preparation of Aptamer-Coated Gold Probes

The preparation methods for aptamer-coated gold probes have been described previously in U.S. Provisional patent application entitled “Aptamer-Nanoparticle Conjugates” (Application No. 60/567,874, filed may 3, 2004), which is incorporated herein in its entirety.

(a) 50 nm Diameter Gold Nanoparticles

Solutions of 50 nm diameter gold particles were purchased from Ted Pella, Inc. for the described experiments.

(b) Synthesis of Steroid Disulfide Modified Oligonucleotides (SDO) as Aptamers

The procedure for synthesizing SDO's is described above. An anti-IgE aptamer and T₂₀ diluent were synthesized using this procedure. The anti-IgE aptamer sequence is reported to have a high binding affinity for human IgE.^{43,44}

| | |
|---|---|
| 5′Steroid-AAA AAA AAA A-CGC GGG GCA | SEQ ID NO 25 |
| CGT TTA TCC GTC CCT CCT AGT GGC GTG | |
| CCC CGC GC 3′.: | |
| 5′ Steroid-TTT TTT TTT TTT TTT TT: | SEQ ID NO 26 |

(c) Attachment of SDOs to 50 nm Diameter Gold Particles

Solutions of 50 nm diameter gold particle were used as delivered from Ted Pella, Inc. The gold nanoparticle probes were prepared by loading the gold particles with steroid disulfide modified oligonucleotides using a modification of a previously developed literature procedure.¹⁵ Briefly, the anti-IgE aptamer (0.9 μM final concentration) and A₂₀ diluent sequence (1.8 μM final concentration) were initially incubated with the gold nanoparticles for >16 hours. Next, sodium dodecyl sulfate (SDS) detergent was added to a final concentration of 0.01%, followed by successive additions of NaCl to a final concentration of 0.8 M NaCl.⁴⁵ The aptamer-modified particles were isolated by centrifugation (2300 rcf for 30 minutes), washed in an equivalent amount of water, and then redispersed in 10 mM Sodium Phosphate, 0.1 M NaCl, 0.01% azide. All probes were stored at 4° C.

### Example 18

### Preparation of Aptamer-Coated Gold Probe Arrays

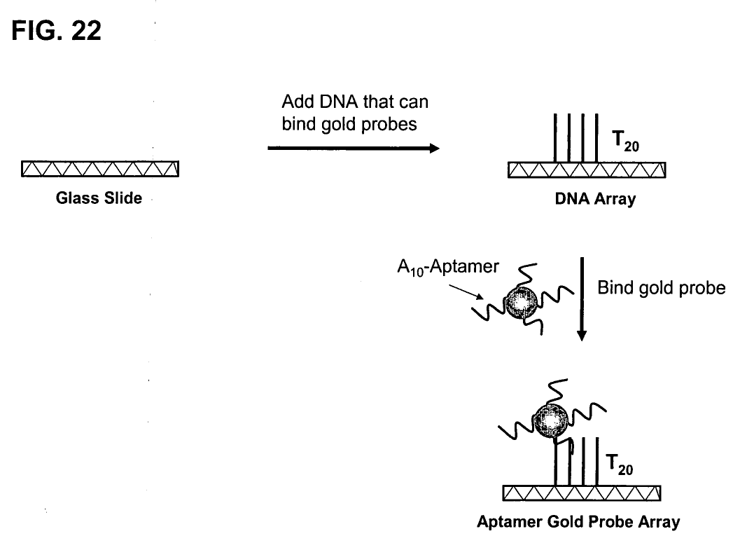

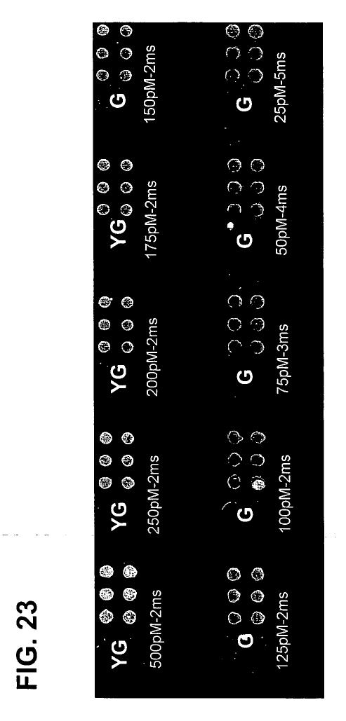

The aptamer coated gold probes were immobilized onto a waveguide substrate through hybridization to an amine modified T₂₀ oligonucleotide (SEQ ID NO: 23) covalently attached to the surface (FIG. 22). Typically, the amine modified T₂₀ oligonucleotides were resuspended in 1×PBS pH 7.2 at a final concentration of 500 uM and arrayed onto Codelink slides (Amersham, Inc.) or Superaldehyde slides (Telechem International) using an Affymetrix GMS 417 pin and ring microarrayer equipped with a 500 micron diameter pin. The slides were incubated overnight in a humidity chamber and subsequently washed with 1×PBS (pH 7.2), 0.01% Tween 20 buffer. Typically, the oligonucleotides were arrayed in triplicate in two rows, and ten replicates of the arrayed spots were produced on each slide. The arrays were partitioned into separate test wells using silicone gaskets (Grace Biolabs). The aptamer-modifed gold probes (sequence A10-aptamer1 with an T₂₀ diluent, SEQ ID NO: 24) were then added at various concentrations in a second step for 15 minutes at room temperature (1×PBS, 1 mM MgCl₂, 0.01% Tween20) to form the ‘aptamer coated gold probe arrays’. Each probe array was illuminated with white light and then imaged with a color CCD camera to record the scatter color. As shown in FIG. 23, the AGPs were immobilized onto the Aldehyde substrate at the complementary T₂₀ spots, and the probes scatter predominantly green—greenish/yellow light depending on the probe concentration. This indicates that the density of AGP bound to the surface can be controlled by the amount of gold probe added.

### Example 19

### Preparation of Antibody-Coated Gold Probes

Goat polyclonal anti-IgE antibodies were purchased from Chemicon International Inc. (Temecula, Calif.). Gold nanoparticles (50 nm diameter) were purchased from Ted Pella, Inc (Redding, Calif.). The anti-IgE antibodies were attached to gold nanoparticles via direct binding of the antibody to the gold particle. The procedure was adapted from an existing protocol developed by British Biocell International. Briefly, the 50 nm diameter gold particles were adjusted to a pH between 9-10 using sodium carbonate buffer. 3 μg of the antibody was added per milliliter of gold nanoparticle and incubated at room temperature for 1.0 hour. Next, the probes were filtered through 0.2 um diameter cellulose acetate filters, and then BSA (10% w/w solution) was added to a final concentration of 1% to stabilize the particles. The antibody-gold nanoparticle conjugates were centrifuged at 2100 G for 25 minutes, and the supernatant was removed leaving a reddish particle precipitate at the bottom of the eppendorf tube. The particles were redispersed in buffer (20 mM Tris-HCl (pH 8.5), 0.1% BSA and 0.01% azide), and the UV-visible absorbance was measured to determine the final nanoparticle concentration using an estimated extinction coefficient of ε_{lambdamax}=1.5×10¹⁰ M⁻¹cm⁻¹ for the 50 nm diameter gold particles.

It should be noted that the probes were brought to 0.2 M NaCl, and the amount of probe aggregation was measured by analyzing the scatter color (a scatter color of green indicates probe stability) on a waveguide substrate prior to the BSA step. This ensures that the probes are stable and sufficient antibody is attached during the preparation process.

### Example 20

### Human IgE Detection on Anti-IgE Aptamer Coated Gold Probe Arrays

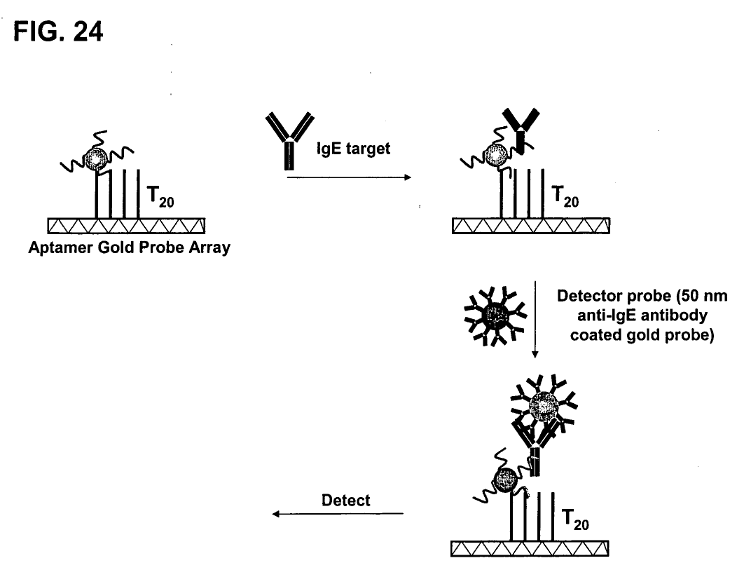

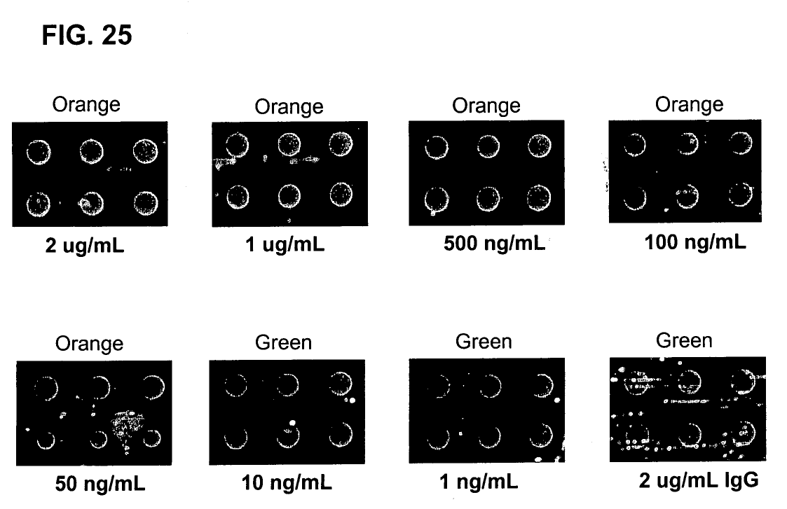

The detection of human IgE target was tested on the anti-IgE aptamer coated gold probe arrays (FIG. 24). For these studies, the anti-IgE aptamer coated gold probes (75 pM) were immobilized on the T₂₀ arrays as describe above. All assay steps were performed at room temperature in 40 μL reaction volumes. In the first step, different concentrations of human IgE (2 ug/mL-1 ng/mL) or human IgG as a negative control (2 ug/mL) were incubated on separate test arrays for 30 minutes in 1 mM MgCl₂, 1×PBS, 0.01% Tween20. In the second step, anti-IgE antibody coated gold probes (prepared as described above) were incubated on the array for 10 minutes at a probe concentration of 450 pM in a buffer containing 1 mM MgCl₂, 1×PBS, 0.01% Tween20, 2% dextran sulfate. The scatter color from each probe array was recorded using a color CCD camera after illumination with white light (FIG. 25). A change in scatter color from green to orange was observed for samples containing >50 ng/mL of IgE target (2.5 ng total IgE). Samples containing <10 ng/mL of IgE target or 2 μg/mL of IgG target remained green in scatter color. It should be noted that the scatter color can also be detected visually with the naked eye.

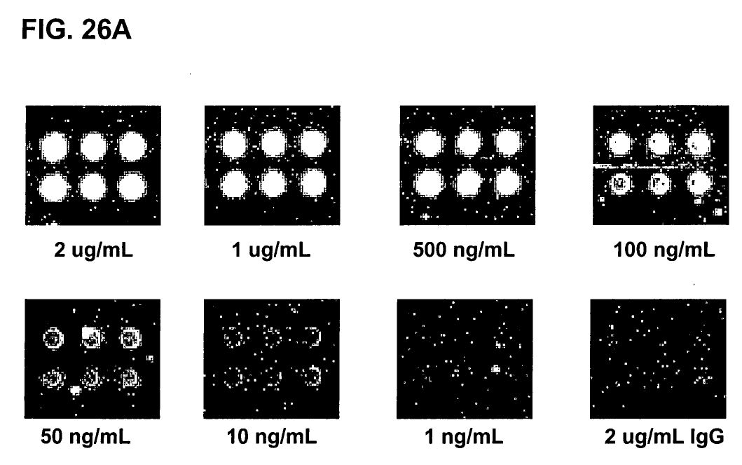

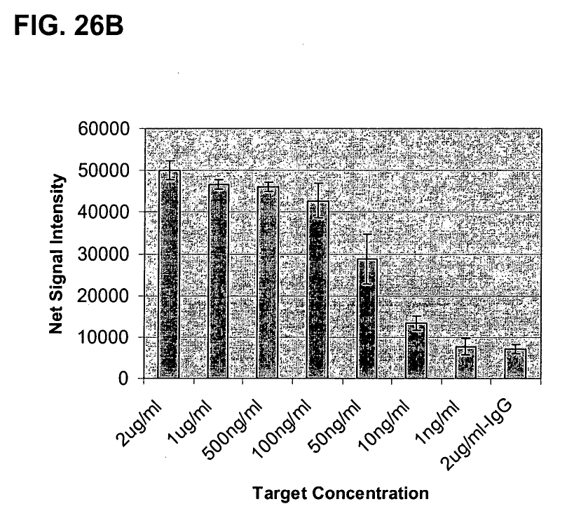

In an alternative method of analysis, the slide was washed in a 5% formamide, 1% Tween 20 prior to imaging with the Verigene ID detection system (FIG. 26). The washing step reduced the amount of green scatter from the human IgG control sample while increasing the colorimetric red-shift in scatter observed for the human IgE target samples. This indicated that the aptamer coated gold probes may be removed from the array by dehybridization in the wash step while AGP probe complexes formed from human IgE target and anti-IgE antibody coated gold probe remain attached to the slide. Therefore, background signal due to unbound probes (green scatter) may be selectively removed via a simple washing process. It should be noted that this effect was first observed after washing the slides with water, and subsequent experiments using different concentrations of formamide and tween demonstrated that 5% formamide, 1% Tween 20 produced the best removal of background while retaining signal from gold probe complexes. The net signal intensity from a 10 ng/mL sample of human IgE was >3 standard deviations over the net signal intensity of a human IgG negative control sample. This represented at least a 5 fold improvement in detection limit over visually analyzing the color change (˜50 ng/mL limit of detection), and it demonstrated that the AGP arrays can be imaged with the Verigene ID detection system in conjunction with a wash step.

### Example 21

### Preparation of Nanoparticle-Oligonucleotide Conjugate Probes

In this Example, a representative nanoparticle-oligonucleotide conjugate detection probe was prepared for use in the detection of surface immobilized nucleic acid targets.

(a) 60 nm Diameter Gold Nanoparticles

Solutions of 60 nm diameter gold particles were purchased from Ted Pella, Inc. for the described experiments.

(b) Synthesis of Steroid Disulfide Modified Oligonucleotides (SDO)

Oligonucleotides were synthesized on a 1-micromole scale using an Applied Biosystems Expedite 8909 DNA synthesizer in single column mode using phosphoramidite chemistry. Eckstein, F. (ed.) Oligonucleotides and Analogues: A Practical Approach (IRL Press, Oxford, 1991). All synthesis reagents were purchased from Glen Research or Applied Biosystems. Average coupling efficiency varied from 98 to 99.8%, and the final dimethoxytrityl (DMT) protecting group was removed from the oligonucleotides so that the steroid disulfide phosphoramidite could be coupled.

To generate 5′-terminal steroid-cyclic disulfide oligonucleotide derivatives (see Letsinger et al., 2000, Bioconjugate Chem. 11:289-291 and PCT/US01/01190 (Nanosphere, Inc.), the disclosure of which is incorporated by reference in its entirety), the final coupling reaction was carried out with a cyclic dithiane linked epiandrosterone phosphoramidite on Applied Biosystems automated Expedite 8909 synthesizer, a reagent that prepared using trans 1,2-dithiane-4,5-diol, epiandrosterone and p-toluenesulphonic acid (PTSA) in presence of toluene. The phosphoramidite reagent may be prepared as follows: a solution of epiandrosterone (0.5 g), trans 1,2-dithiane-4,5-diol (0.28 g), and p-toluenesulfonic acid (15 mg) in toluene (30 mL) was refluxed for 7 h under conditions for removal of water (Dean Stark apparatus); then the toluene was removed under reduced pressure and the reside taken up in ethyl acetate. This solution was washed with 5% NaHCO₃, dried over sodium sulfate, and concentrated to a syrupy reside, which on standing overnight in pentane/ether afforded a steroid-dithioketal compound as a white solid (400 mg); Rf (TLC, silica plate, ether as eluent) 0.5; for comparison, Rf values for epiandrosterone and 1,2-dithiane-4,5-diol obtained under the same conditions are 0.4, and 0.3, respectively. The compound was purified by column chromatography. Subsequently, recrystallization from pentane/ether afforded a white powder, mp 110-112° C.; ¹H NMR, δ 3.6 (1H, C³OH), 3.54-3.39 (2H, m 2OCH of the dithiane ring), 3.2-3.0 (4H, m 2CH₂S), 2.1-0.7 (29H, m steroid H); mass spectrum (ES⁺) calcd for C₂₃H₃₆O₃S₂ (M+H) 425.2179, found 425.2151. Anal. (C₂₃H₃₇O₃S₂) S: calcd, 15.12; found, 15.26. To prepare the steroid-disulfide ketal phosphoramidite derivative, the steroid-dithioketal (100 mg) was dissolved in THF (3 mL) and cooled in a dry ice alcohol bath. N,N-diisopropylethylamine (80 μL) and β-cyanoethyl chlorodiisopropylphosphoramidite (80 μL) were added successively; then the mixture was warmed to room temperature, stirred for 2 h, mixed with ethyl acetate (100 mL), washed with 5% aq. NaHCO₃ and with water, dried over sodium sulfate, and concentrated to dryness. The residue was taken up in anhydrous acetonitrile and then dried under vacuum; yield 100 mg; ³¹P NMR 146.02. The epiandrosterone-disulfide linked oligonucleotides were synthesized on Applied Biosystems Expedite 8909 gene synthesizer without final DMT removal. After completion, epiandrosterone-disulfide linked oligonucleotides were deprotected from the support under aqueous ammonia conditions and purified on HPLC using reverse phase column.

Reverse phase HPLC was performed with a Dionex DX500 system equipped with a Hewlett Packard ODS hypersil column (4.6×200 mm, 5 mm particle size) using 0.03 M Et₃NH⁺ OAc⁻ buffer (TEAA), pH 7, with a 1 mL/min. gradient of 95% CH₃CN/5% TEAA. The flow rate was 1 mL/min. with UV detection at 260 nm. Preparative HPLC was used to purify the DMT-protected unmodified oligonucleotides. After collection and evaporation of the buffer, the DMT was cleaved from the oligonucleotides by treatment with 80% acetic acid for 30 min. at room temperature. The solution was then evaporated to near dryness, water was added, and the cleaved DMT was extracted from the aqueous oligonucleotide solution using ethyl acetate. The amount of oligonucleotide was determined by absorbance at 260 nm, and final purity assessed by reverse phase HPLC.

(c) Attachment of SDOs to 60 nm Diameter Gold Particles

Solutions of 60 nm diameter gold particles were used as delivered from Ted Pella, Inc. The gold nanoparticle probes were prepared by loading the gold particles with steroid disulfide modified oligonucleotides using a modification of a previously developed literature procedure¹⁵. Briefly, 8 nmol of SDO was added per 3 mL of gold nanoparticle and incubated for 15 hours at room temperature. After 24 hours, aqueous sodium dodecyl sulfate (SDS, 10% by weight) was added to the solution (final concentration: 0.01%). Then, aqueous 2 M NaCl was added to a final concentration of 0.1 M NaCl. After standing for 4 additional hours, the NaCl concentration was increased to 0.2 M. This was repeated the following day to bring the NaCl concentration of the probe solution to 0.3 M and again 4 hours later to bring the final concentration to 0.5 M. After an additional overnight incubation, the salt was raised to 0.8 M and incubated for an additional four hours before the the SDO-gold nanoparticle conjugates were isolated with a Beckman Coulter Microfuge 18 by centrifugation (2100 rcf for 15 minutes). After centrifugation, a dark red gelatinous residue remained at the bottom of the eppendorf tube. The supernatant was removed, and the conjugates were washed (2×) with 0.1 M NaCl, 10 mM phosphate (pH 7) (original colloid volume) and redispersed in 20 mM Tris HCL (pH 7).

The following nanoparticle-oligonucleotide conjugates specific for segments of the Human Coagulation Factor V gene were prepared in this manner:

| | |
|---|---|
| Probe 1: | |
| gold-[S′-5′-A₁₀-TGGACAGGCGAGGAATAC | (SEQ ID NO: 20) |
| AG3′]ₙ | |
| Probe 2: | |
| gold-[S′-5′-TGATGCCCAGTGCTTAACAAGA | (SEQ ID NO: 21) |
| CCATACTACAGTG3′]ₙ | |

### Example 22

### Molecular Weight Dependence of the Function Of Dextran Sulfate

The target sequences used for this study is as follows:

| | |
|---|---|
| DNA Target: | |
| 5′CTTATAAGTGGAACATCTTAGAGTTTGATGAA | (SEQ ID NO: 22) |
| CCCACAGAAAATGATGCCCAGTGCTTAACAAGAC | |
| CATACTACAGTGACGTGGACATCATGAGAGACAT | |
| CGCCTCTGGGCTAATAGGACTACTTCTAATCTGT | |
| AAGAGCAGATCCCTGGACAGGCGAGGAATACAGG | |
| TATTTTGTCCTTGAAGTAACCTTTCAGAAATTCT | |
| GAGAATTTCTTCTGGCTAGAACATGTTAGGTCTC | |
| CTGGCTAAATAATG3′ | |
| Probe 1: | |
| gold-[S′-5′-A₁₀-TGGACAGGCGAGGAATAC | (SEQ ID NO: 20) |
| AG3′]ₙ | |
| Probe 2: | |
| gold-[S′-5′-TGATGCCCAGTGCTTAACAAGA | (SEQ ID NO: 21) |
| CCATACTACAGTG3′]ₙ | |

Dextran sulfate sodium salt from Leuconostoc ssp. of average molecular weight of ˜100,000, ˜500,000, and ˜1,000,000 was purchased from Fluka Biochemika. Dextran sulfate sodium salt from Leuconostoc ssp. of average molecular weight of ˜10,000 was purchased from Sigma Chemical Company.

The nucleic acid target (SEQ ID NO: 22) was amplified via PCR and stored at 4° C. when not in use. Steroid disulfide modified oligonucleotides (SEQ ID NO: 20 and SEQ ID NO: 21) complementary to the nucleic acid target were conjugated to 60 nm diameter gold particles as described above to produce gold probes 1 and 2. Both probes 1 and 2 (SEQ ID NO: 20 and SEQ ID NO: 21, respectively) were diluted to 100 pM with 20 mM Tris at pH 7.

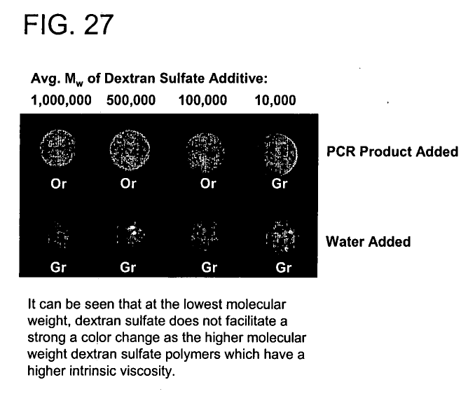

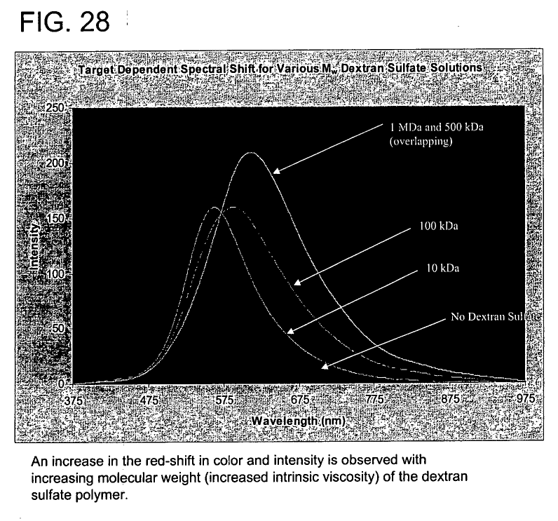

In several 0.5 mL μcentrifuge tubes test samples were prepared. Test samples were comprised of 3 μL of a solution containing a 1:1 ratio of Probe 1 and Probe 2, 3 μL of a solution containing target (SEQ ID NO: 22) at 30 μM or water as noted, 4 μL of solution containing 18.75% v/v formamide and 3.75 mM MgCl₂, and 5 μL of a solution containing 12% w/v of dextran sulfates of varying molecular weights. The solutions were heated to 95° C. for 30 sec. and allowed to incubate at room temperature for 20 min. 1 μL of each solution was then spotted on poly-1-lysine treated glass and illuminated via planar waveguide and imaged, FIG. 27. A green scatter color was observed for each solution that did not contain target. A substantial change in scatter color from green to orange was observed for the samples containing dextran sulfate of molecular weight of 100 kDa or above. Using dextran sulfate of M_{w} 10 kDa, a very slight color change to green/greenish yellow was observed. The samples also were imaged in solution after white light illumination using a diode array detector, FIG. 28. The diode array detector provides both intensity and color of scattered light at a higher spectral resolution than the color CMOS detector. With the diode array detector, larger intensity increases and plasmon frequency red-shifts are observed as the molecular weight of dextran sulfate increases from 10,000 to 500,000. This data demonstrates significant molecular weight dependence for the function of dextran sulfate in the assay.

### Example 23

### Utilization of Neutral Polysacharides as Volume Exclusion Reagents

The target sequences used for this study is as follows:

| | |
|---|---|
| DNA Target: | |
| 5′CTTATAAGTGGAACATCTTAGAGTTTGATGAA | (SEQ ID NO: 22) |
| CCCACAGAAAATGATGCCCAGTGCTTAACAAGAC | |
| CATACTACAGTGACGTGGACATCATGAGAGACAT | |
| CGCCTCTGGGCTAATAGGACTACTTCTAATCTGT | |
| AAGAGCAGATCCCTGGACAGGCGAGGAATACAGG | |
| TATTTTGTCCTTGAAGTAACCTTTCAGAAATTCT | |
| GAGAATTTCTTCTGGCTAGAACATGTTAGGTCTC | |
| CTGGCTAAATAATG3′ | |
| Probe 1: | |
| gold-[S′-5′-A₁₀-TGGACAGGCGAGGAATAC | (SEQ ID NO: 20) |
| AG3′]ₙ | |
| Probe 2: | |
| gold-[S′-5′-TGATGCCCAGTGCTTAACAAGA | (SEQ ID NO: 21) |
| CCATACTACAGTG3′]ₙ | |

Dextran polymer from Leuconostoc mesenteroides of average molecular weight of 500,000 was purchased from Sigma Chemical Company.

The nucleic acid target (SEQ ID NO: 22) was amplified via PCR and stored at 4° C. when not in use. Steroid disulfide modified oligonucleotides (SEQ ID NO: 20 and SEQ ID NO: 21) complementary to the nucleic acid target were conjugated to 60 nm diameter gold particles as described above to produce gold probes 1 and 2. Both probes 1 and 2 (SEQ ID NO: 20 and SEQ ID NO: 21, respectively) were diluted to 100 pM with 20 mM Tris at pH 7.

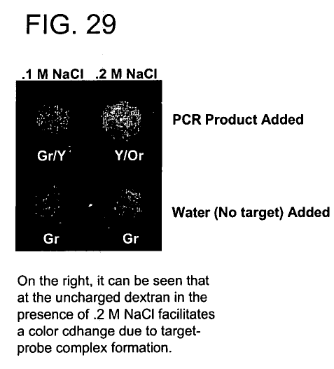

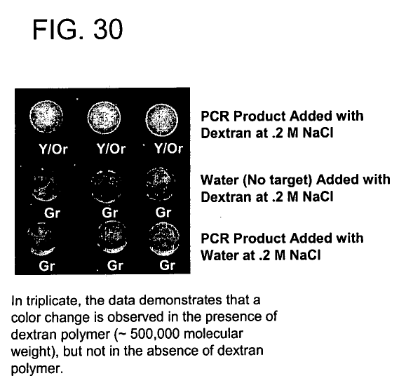

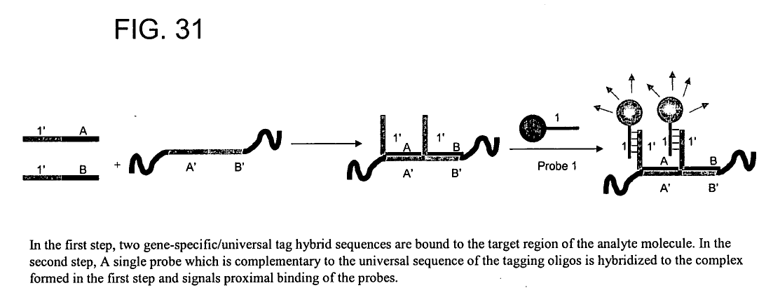

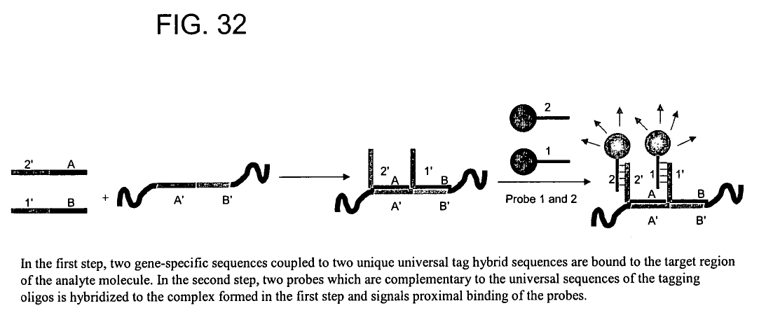

In several 0.5 mL μL μcentrifuge tubes test samples were prepared. Test samples were comprised of 3 μL of a solution containing a 1:1 ratio of Probe 1 and Probe 2, 3 μL of a solution containing target (SEQ ID NO: 22) at 30 nM or water as noted, 4 μL of solution containing 18.75% v/v formamide and 3.75 mM MgCl₂, 2.5 μL of a solution containing 24% w/v of unionized dextran molecular weight 500 kDa, and either 1 or 2 μL of a solution of 1.66 M NaCl and 1.5 μL or 0.5 μL of additional water, respectively. The solutions were heated to 95° C. for 30 sec. and allowed to incubate at room temperature for 20 min. 1 μL of each solution was then spotted on poly-1-lysine treated glass and illuminated via a planar waveguide and imaged, FIG. 29. A green scatter color was observed in control solutions that did not contain target. A target-based change in scatter color from green to greenish-yellow or yellow-orange was observed for the samples containing dextran polymer and 0.1 M or 0.2 M NaCl, respectively. This data demonstrates that neutral polysaccharides may be used in the methods of the invention. As a final demonstration, the samples that demonstrated a color change at 0.2 M NaCl were run in triplicate with additional controls. A green-to-orange color shift is only observed in the presence of target, salt, and dextran polymer, FIG. 30.

### Example 24

Application of Surface Plasmon Resonance to the Development of Novel Probes for High Resolution In Situ Hybridization and In Situ Staining with Aptamer or Antibody Functionalized Probes.

Of particular advantage in in situ hybridization, is the concept of generating a unique color through surface plasmon resonance. By designing two probes such that they have to bind close to each other near or at the target site, these specifically bound probes will generate a color that is distinctly different from non-specifically bound probes, or probes that cross-react with other sequences in the metaphase spread, since it is unlikely that two probes will bind next to each other non-specifically. Examples herein have demonstrated that two or more gold nanoparticle probes may be used to detection specific nucleic acid sequences in complex genomic DNA samples by measuring changes in colorimetric scatter homogeneously (i.e. no separation of bound or unbound particles). Furthermore, examples herein have demonstrated that individual gold nanoparticle complexes bound to specific nucleic acid sequences immobilized on a glass surface can be detected and differentiated on the basis of scatter color from single gold nanoparticles hybridized to the same nucleic acid target using evanescent illumination in conjunction with a >10× microscope objective and color CCD camera. Thus, short oligonucleotide probes attached to metal nanoparticles >30 nm diameter can be targeted to specific nucleic acid sequences, including but not limited to, SNP sites, sequence repeats, insertions, deletions, or other sequence aberrations by in situ hybridization. This would increase the resolution of FISH by 4-5 orders of magnitude and allow the development of probes for some 4,000 genetic diseases (metabolic disorders) and many more SNP's that are all characterized by point mutations. This method is particularly useful when probes are targeted at free chromatin, DNA fibers or mechanically stretched chromosomes³³, but is also applicable to regular metaphase spreads.

Moreover, this concept can be easily extended to the use of nanoparticle probes that are functionalized with aptamers or antibodies. Binding of two of these probes in close proximity on a target molecule, such as DNA, protein, lipid, carbohydrate, or combinations and complexes thereof, will result in the interaction of the surface plasmons of these probes, resulting in a unique scatter color, which differentiates the probes bound specifically to the target from probes that bind to non-target molecules or are merely sticking to surfaces and present general background.

### Example 25

### Preparation of Nanoparticle-Oligonucleotide Conjugate Probes

In this Example, a representative nanoparticle-oligonucleotide conjugate detection probe was prepared for use in the detection of universal tagged gene-specific sequences.

(a) 50 nm Diameter Gold Nanoparticles

Solutions of 50 nm diameter gold particles were purchased from Ted Pella, Inc. for the described experiments.

(b) Synthesis of Steroid Disulfide Modified Oligonucleotides (SDO)

Oligonucleotides were synthesized on a 1 micromole scale using a Applied Biosystems Expedite 8909 DNA synthesizer in single column mode using phosphoramidite chemistry. Eckstein, F. (ed.) Oligonucleotides and Analogues: A Practical Approach (IRL Press, Oxford, 1991). All synthesis reagents were purchased from Glen Research or Applied Biosystems. Average coupling efficiency varied from 98 to 99.8%, and the final dimethoxytrityl (DMT) protecting group was removed from the oligonucleotides so that the steroid disulfide phosphoramidite could be coupled.

To facilitate hybridization of the probe sequence with the target, a deoxyadenosine oligonucleotide-polyethylene glycol (dA₁₀-PEG) was included on the 5′ end in the probe sequence as a spacer.

To generate 5′-terminal steroid-cyclic disulfide oligonucleotide derivatives (see Letsinger et al., 2000, Bioconjugate Chem. 11:289-291 and PCT/US01/01190 (Nanosphere, Inc.), the disclosure of which is incorporated by reference in its entirety), the final coupling reaction was carried out with a cyclic dithiane linked epiandrosterone phosphoramidite on Applied Biosystems automated Expedite 8909 synthesizer, a reagent that prepared using trans 1,2-dithiane-4,5-diol, epiandrosterone and p-toluenesulphonic acid (PTSA) in presence of toluene. The phosphoramidite reagent may be prepared as follows: a solution of epiandrosterone (0.5 g), trans 1,2-dithiane-4,5-diol (0.28 g), and p-toluenesulfonic acid (15 mg) in toluene (30 mL) was refluxed for 7 h under conditions for removal of water (Dean Stark apparatus); then the toluene was removed under reduced pressure and the reside taken up in ethyl acetate. This solution was washed with 5% NaHCO₃, dried over sodium sulfate, and concentrated to a syrupy reside, which on standing overnight in pentane/ether afforded a steroid-dithioketal compound as a white solid (400 mg); Rf (TLC, silica plate, ether as eluent) 0.5; for comparison, Rf values for epiandrosterone and 1,2-dithiane-4,5-diol obtained under the same conditions are 0.4, and 0.3, respectively. The compound was purified by column chromatography. Subsequently, recrystallization from pentane/ether afforded a white powder, mp 110-112° C.; ¹H NMR, δ 3.6 (1H, C³OH), 3.54-3.39 (2H, m 2OCH of the dithiane ring), 3.2-3.0 (4H, m 2CH₂S), 2.1-0.7 (29H, m steroid H); mass spectrum (ES⁺) calcd for C₂₃H₃₆O₃S₂ (M+H) 425.2179, found 425.2151. Anal. (C₂₃H₃₇O₃S₂) S: calcd, 15.12; found, 15.26. To prepare the steroid-disulfide ketal phosphoramidite derivative, the steroid-dithioketal (100 mg) was dissolved in THF (3 mL) and cooled in a dry ice alcohol bath. N,N-diisopropylethylamine (80 μL) and β-cyanoethyl chlorodiisopropylphosphoramidite (80 μL) were added successively; then the mixture was warmed to room temperature, stirred for 2 h, mixed with ethyl acetate (100 mL), washed with 5% aq. NaHCO₃ and with water, dried over sodium sulfate, and concentrated to dryness. The residue was taken up in anhydrous acetonitrile and then dried under vacuum; yield 100 mg; ³¹P NMR 146.02. The epiandrosterone-disulfide linked oligonucleotides were synthesized on Applied Biosystems Expedite 8909 gene synthesizer without final DMT removal. After completion, epiandrosterone-disulfide linked oligonucleotides were deprotected from the support under aqueous ammonia conditions and purified on HPLC using reverse phase column.

Reverse phase HPLC was performed with a Dionex DX500 system equipped with a Hewlett Packard ODS hypersil column (4.6×200 mm, 5 mm particle size) using 0.03 M Et₃NH⁺ OAc⁻ buffer (TEAA), pH 7, with a 1 mL/min. gradient of 95% CH₃CN/5% TEAA. The flow rate was 1 mL/min. with UV detection at 260 nm. Preparative HPLC was used to purify the DMT-protected unmodified oligonucleotides. After collection and evaporation of the buffer, the DMT was cleaved from the oligonucleotides by treatment with 80% acetic acid for 30 min. at room temperature. The solution was then evaporated to near dryness, water was added, and the cleaved DMT was extracted from the aqueous oligonucleotide solution using ethyl acetate. The amount of oligonucleotide was determined by absorbance at 260 nm, and final purity assessed by reverse phase HPLC.

(c) Attachment of SDOs to 50 nm Diameter Gold Particles

Solutions of 50 nm diameter gold particle were used as delivered from Ted Pella, Inc. The gold nanoparticle probes were prepared by loading the gold particles with steroid disulfide modified oligonucleotides using a modification of a previously developed literature procedure¹⁵. Briefly, 8 nmol of SDO was added per 3 mL of gold nanoparticle and incubated for 15 hours at room temperature. After 24 hours, aqueous sodium dodecyl sulfate (SDS, 10% by weight) was added to the solution (final concentration: 0.01%). Then, aqueous 2 M NaCl was added to a final concentration of 0.1 M NaCl. After standing for 24 additional hours, the NaCl concentration was increased to 0.2 M. This was repeated the following day to bring the NaCl concentration of the probe solution to 0.3 M. After 24 additional hours, the SDO-gold nanoparticle conjugates were isolated with a Beckman Coulter Microfuge 18 by centrifugation (5000 rpm for 25 minutes for 30 nm, 3000 rpm for 15 minutes for 40 nm, 3000 rpm for 15 minutes for 50 nm). After centrifugation, a dark red gelatinous residue remained at the bottom of the eppendorf tube. The supernatant was removed, and the conjugates were washed (2×) with 0.1 M NaCl, 10 mM phosphate (pH 7) (original colloid volume) and redispersed in 20 mM Tris HCL (pH 7).

The following nanoparticle-oligonucleotide conjugates specific for segments of the mecA gene were prepared in this manner:

Probe 1:

gold-[S′-5′-A₃₀ 3′]ₙ (SEQ ID NO: 25)

### EXAMPLE 26

### Changes in Scatter Color Based on Universally Tagged Gene-Specific Linkers

The target sequences used for this study is as follows:

| | |
|---|---|
| DNA Target: | |
| 5′TGGTGAAGTTGTAATCTGGAACTTGTTGAGCA | (SEQ ID NO: 26) |
| GAGGTTCTTTTTTATCTTCGGTTAATTTATTATA | |
| TTCTTCGTTACTCATGCCAT3′ | |
| 5′TTCCAGATTACACTTCACCATTTTTTTTTTTT | (SEQ ID NO: 27) |
| TTTTTTTT3′ | |
| 5′AAAGAACCTCTGCTCAACAAGTTTTTTTTTTT | (SEQ ID NO: 28) |
| TTTTTTTTT3′ | |

The nucleic acid target (Sequence ID NO: 26) was purchased from IDT and suspended in a pure water at a concentration of ˜100 nM. Steroid disulfide modified oligonucleotides (SEQ ID NO: 25) complementary to the nucleic acid target were conjugated to 50 nm diameter gold particles as described above to produce the gold probe 1 which was diluted to 100 pM with 20 mM Tris at pH 7.

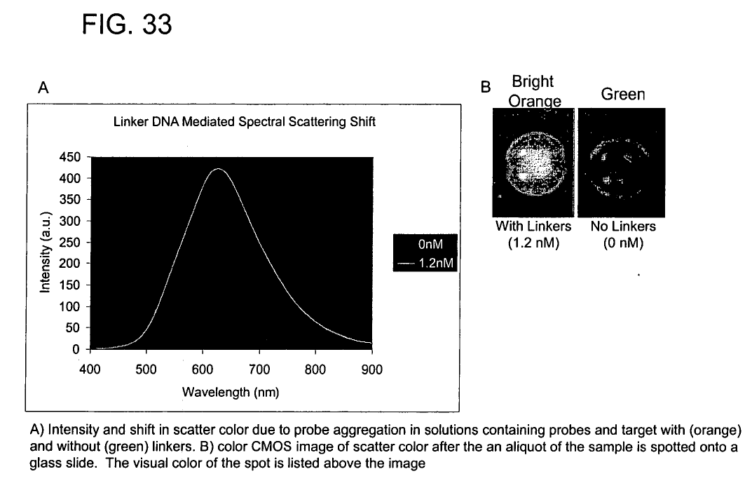

In two separate 0.5 mL μcentrifuge tubes test samples were prepared. Test samples were comprised of 4 μL of a solution containing Probe 1, 3.5 μL of a solution containing the DNA target (Sequence ID NO: 2) at ˜100 nM, 4 μL of a hybridization solution containing 18.75% v/v formamide, 3.75 mM MgCl₂, and 16.25% dextran sulfate, an intermediate oligonucleotide linker solution containing 10 nM of each oligos 1 and 2 (SEQ ID NO: 27 and 28) or water as noted, and finally 1.75 μL of water. The solutions were heated to 95° C. for 30 sec. and allowed to incubate at room temperature for 1 hr. The samples were imaged via two methods. First, the colorimetric scatter from each sample was recorded in a cuvette using a diode array detector after illumination with white light (Ocean Optics, Inc.). The colorimetric scatter was recorded at 90 degrees, FIG. 33A. Second, 1 μL of each solution was then spotted on poly-1-lysine treated glass and illuminated via planar waveguide and imaged, FIG. 33B. A green scatter color was observed for samples containing 50 nm gold particles and the nucleic acid target without the intermediate oligonucleotide linkers. A change in scatter color from green to orange was observed for nucleic acid target samples containing both the intermediate oligonucleotide linkers and the gold probes. This example demonstrates the feasibility of using an intermediate oligonucleotide linker in homogeneous detection assays that monitor changes in colorimetric scatter.

It should be understood that the foregoing disclosure emphasizes certain specific embodiments of the invention and that all modifications or alternatives equivalent thereto are within the spirit and scope of the invention as set forth in the appended claims.

### REFERENCES

(1) Kirk, B. W.; Feinsod, M.; Favis, R.; Kliman, R. M.; Barany, F. Nucleic Acids Research 2002, 30, 3295-3311.
(2) Whelen, A. C.; Persing, D. H. Annual Review of Microbiology 1996, 50, 349-373.
(3) Bernard, P. S.; Wittwer, C. T. Clinical Chemistry 2002, 48, 1178-1185.
(4) Kwok, P. Y. Annual Review of Genomics and Human Genetics 2001, 2, 235-258.
(5) Mackay, I. M.; Arden, K. E.; Nitsche, A. Nucleic Acids Research 2002, 30, 1292-1305.
(6) Hall, J. G.; Eis, P. S.; Law, S. M.; Reynaldo, L. P.; Prudent, J. R.; Marshall, D. J.; Allawi, H. T.; Mast, A. L.; Dahlberg, J. E.; Kwiatkowski, R. W.; Arruda, M.; Neri, B. P.; Lyamichev, V. I. Proceedings of the National Academy of Sciences of the United States of America 2000, 97, 8272-8277.
(7) Jenison, R.; Yang, S.; Haeberli, A.; Polisky, B. Nature Biotechnology 2001, 19, 62-65.
(8) Mirkin, C. A.; Letsinger, R. L.; Mucic, R. C.; Storhoff, J. J. Nature 1996, 382, 607-609.
(9) Elghanian, R.; Storhoff, J. J.; Mucic, R. C.; Letsinger, R. L.; Mirkin, C. A. Science 1997, 277, 1078-1081.
(10) Storhoff, J. J.; Lazarides, A. A.; Mucic, R. C.; Mirkin, C. A.; Letsinger, R. L.; Schatz, G. C. Journal of the American Chemical Society 2000, 122, 4640-4650.
(11) Thanh, N. T. K.; Rosenzweig, Z. Analytical Chemistry 2002, 74, 1624-1628.
(12) Otsuka, H.; Akiyama, Y.; Nagasaki, Y.; Kataoka, K. Journal of the American Chemical Society 2001, 123, 8226-8230.
(13) Kim, Y. J.; Johnson, R. C.; Hupp, J. T. Nano Letters 2001, 1, 165-167.
(14) Liu, J.; Lu, Y. Journal of the American Chemical Society 2003, 125, 6642-6643.
(15) Storhoff, J. J.; Elghanian, R.; Mucic, R. C.; Mirkin, C. A.; Letsinger, R. L. Journal of the American Chemical Society 1998, 120, 1959-1964.
(16) Lazarides, A. A.; Schatz, G. C. Journal of Physical Chemistry B 2000, 104, 460-467.
(17) Reynolds, R. A.; Mirkin, C. A.; Letsinger, R. L. Journal of the American Chemical Society 2000, 122, 3795-3796.
(18) Macbeath, G.; Schreiber, S. L. Science 2000, 289, 1760-1763.
(19) Moody, M. D.; Van Arsdell, S. W.; Murphy, K. P.; Orencole, S. F.; Burns, C. Biotechniques 2001, 31, 186-194.
(20) Nielsen, U. B.; Geierstanger, B. H. Journal Immunol. Meth. 2004, 290, 107-120.
(21) Li, Y. L.; Reichert, W. M. Langmuir 2003, 19, 1557-1566.
(22) Grubisha, D. S.; Lipert, R. J.; Park, H.-Y.; Driskell, J.; Porter, M. D. Anal. Chem. 2003, 75, 5936-5943.
(23) Schweitzer, B.; Wiltshire, S.; Lambert, J.; O'Malley, S.; Kukanskis, K.; Zhu, Z.; Kingsmore, S. F.; Lizardi, P. M.; Ward, D.C. Proceedings of the National Academy of Sciences of the United States of America 2000, 97, 10113-10119.
(24) Schweitzer, B.; Roberts, S.; Grimwade, B.; Shao, W.; Wang, M.; Fu, Q.; Shu, Q.; Laroche, I.; Zhou, Z.; Tchernev, V. T.; Christiansen, J.; Velleca, M.; Kingsmore, S. F. Nat. Biotechnol. 2002, 20, 359-365.
(25) Jayasena, S. D. Clin. Chem. 1999, 45, 1628-1650.
(26) Storhoff, J. J.; Mirkin, C. A. Chemical Reviews 1999, 99, 1849-1862.
(27) Niemeyer, C. M. Angew. Chem. Int. Ed. 2001, 40, 4128-4158.
(28) Taton, T. A.; Mirkin, C. A.; Letsinger, R. L. Science 2000, 289, 1757-1760.
(29) Storhoff, J. J.; Marla, S. M.; Bao, P.; Hagenow, S.; Mehta, H.; Lucas, A.; Garimella, V.; Patno, T.; Buckingham, W.; Cork, W.; Muller, U. Biosens. Bioelectron. 2004, 19, 875-883.
(30) Storhoff, J. J.; Elghanian, R.; Mirkin, C. A.; Letsinger, R. L. Langmuir 2002, 18, 6666-6670.
(31) Taton, T. A.; Mucic, R. C.; Mirkin, C. A.; Letsinger, R. L. Journal of the American Chemical Society 2000, 122, 6305-6306.
(32) Niemeyer, C. M.; Ceyhan, B. Angew. Chem. Int. Ed. 2001, 40, 3585-3688.
(33) Nam, J. M.; Park, S. J.; Mirkin, C. A. Journal of the American Chemical Society 2002, 124, 3820-3821.
(34) Brody, E.; Gold, L. Rev. Mol. Biotech. 2000, 74, 5-13.
(35) Potyrailo, R. A.; Conrad, R. C.; Ellington, A. D.; Hieftje, G. M. Anal. Chem. 1998, 70, 3419-3425.
(36) Yguerabide, J.; Yguerabide, E. E. Analytical Biochemistry 1998, 262, 157-176.
(37) Jin, R. C.; Cao, Y. W.; Mirkin, C. A.; Kelly, K. L.; Schatz, G. C.; Zheng, J. G. Science 2001, 294, 1901-1903.
(38) Storhoff, J. J.; Marla, S. M.; Bao, P.; Hagenow, S.; Mehta, H.; Lucas, A.; Garimella, V.; Patno, T.; Buckingham, W.; Cork, W.; Muller, U. Biosensors and Bioelectronics 2003, submitted for publication.
(39) Yguerabide, J.; Yguerabide, E. E. Analytical Biochemistry 1998, 262, 137-156.
(40) Taton, T. A.; Lu, G.; Mirkin, C. A. Journal of the American Chemical Society 2001, 123, 5164-5165.
(41) Wetmur, J. G. Biopolymers 1975, 14, 2517-2524.
(42) Chambers, H. F. Clin. Microbiol. Rev. 1997, 10, 781-791.
(43) Wiegand, T. W.; Williams, P. B.; Dreskin, S. C.; Jouvin, M. H.; Kinet, J. P.; Tasset, D. J. Immunol. 1996, 157, 221-230.
(44) Liss, M.; Petersen, B.; Wolf, H.; Prohaska, E. Anal. Chem. 2002, 74, 4488-4495.
(45) Storhoff, J. J.; Lucas, A. D.; Viswanadham, G.; Bao, Y. P.; Muller, U. Nat. Biotechnol. 2004, 22, 883-887.

1-75. (canceled)

76. A method of detecting for the presence or absence of a single target molecule comprising:
a. providing at least two types of nanoparticles having specific binding complements of a single target molecule attached thereto, the specific binding complements on each type of nanoparticle being capable of recognizing different portions of the single target molecule;
b. forming a light scattering complex by contacting a sample believed to contain the single target molecule with the nanoparticles under conditions effective to allow binding of the specific binding complements to two or more portions of the single target molecule;
c. illuminating the light scattering complex under conditions effective to produce scattered light from said complex; and
d. detecting the light scattered by said light scattering complex as a measure of the presence of the single target molecule.

77. The method of claim 76, wherein single target molecule from the sample is isolated and immobilized on a substrate prior to being contacted with the nanoparticles.

78. The method of claim 76, wherein said illumination step comprises placing at least a portion of the light scattering complex within an evanescent wave of a waveguide.

79. The method of claim 76, wherein said detecting step comprises observing the color of scattered light from the light scattering complex.

80. The method of claim 76, wherein said detecting step comprises observing the intensity of scattered light from the light scattering complex.

81. The method of claim 76, wherein said detecting step comprises observing the color and intensity of scattered light from the light scattering complex.

82. The method of claim 76, wherein said detecting step comprises observing the wavelength and intensity of scattered light from the light scattering complex.

83. The method of claim 76, wherein the nanoparticle is metallic and said detecting step comprises observing a change in surface plasmon band.

84. The method of claim 76, wherein the single target molecule and specific binding complement are complements of a specific binding pair.

85. The method of claim 84, wherein complements of a specific binding pair comprise nucleic acid, oligonucleotide, peptide nucleic acid, polypeptide, antibody, antigen, carbohydrate, protein, peptide, amino acid, hormone, steroid, vitamin, drug, virus, polysaccharides, lipids, lipopolysaccharides, glycoproteins, lipoproteins, nucleoproteins, oligonucleotides, antibodies, immunoglobulins, albumin, hemoglobin, coagulation factors, peptide and protein hormones, non-peptide hormones, interleukins, interferons, cytokines, peptides comprising a tumor-specific epitope, cells, cell-surface molecules, microorganisms, fragments, portions, components or products of microorganisms, small organic molecules, nucleic acids and oligonucleotides, metabolites of or antibodies to any of the above substances.

86. The method of claim 85, wherein nucleic acid and oligonucleotide comprise genes, viral RNA and DNA, bacterial DNA, fungal DNA, mammalian DNA, cDNA, mRNA, RNA and DNA fragments, oligonucleotides, synthetic oligonucleotides, modified oligonucleotides, single-stranded and double-stranded nucleic acids, natural and synthetic nucleic acids, and aptamers.

87. The method of claim 76, wherein the single target molecule is a nucleic acid and the specific binding complement is an oligonucleotide.

88. The method of claim 76, where the single target molecule contains the addition, deletion, transition, transversion, or modification of one or more nucleotides.

89. The method of claim 76, wherein the single target molecule is a protein or antibody and the specific binding complement is an antibody.

90. The method of claim 76, wherein the single target molecule is a carbohydrate, lipid, metabolite, or combination thereof and the specific binding complement is a protein receptor or an antibody.

91. The method of claim 76, wherein the single target molecule is a bacterial cell containing surface antigens, and the specific binding complement is an antibody or aptamer.

92. The method of claim 76, wherein the single target molecule is a protein or antibody and the specific binding complement is a polyclonal antibody.

93. The method of claim 76, wherein the single target molecule is a gene sequence from a chromosome and the specific binding complements are oligonucleotides, the oligonucleotides having a sequence that is complementary to at least a portion of the gene sequence.

94. The method of claim 76, wherein the single target molecule is one or more nucleotide sequence in a metaphase spread and the specific binding complements are oligonucleotides, the oligonucleotides having a sequence that is complementary to at least a portion of the one or more nucleotide sequence.

95. The method of claim 76, wherein the single target molecule is one or more nucleotide sequence in a histological specimen and the specific binding complements are oligonucleotides, the oligonucleotides having a sequence that is complementary to at least a portion of the one or more nucleotide sequence.

96. The method of claim 76, wherein the single target molecule is one or more nucleic acid, protein, lipid, or carbohydrate molecules, or combinations thereof, in a histological specimen or metaphase spread and the specific binding complements are oligonucleotides, antibodies, receptors or a combination thereof.

97. The method of claim 76, wherein the single target molecule is one or more nucleic acid, protein, lipid, or carbohydrate molecules, or combinations thereof, in a specimen of human, plant or animal cells that are mounted on a solid surface, and the specific binding complements are oligonucleotides, antibodies, receptors or a combination thereof.

98. The method of claim 93, wherein the nanoparticles are metallic nanoparticles.

99. The method of claim 93, wherein the metallic nanoparticles are gold nanoparticles.

100. The method of claim 93, wherein the nanoparticle is a core-shell particle.

101. The method of claim 93, wherein the wave guide comprises glass, quartz, or plastic.

102. A method of detecting for the presence or absence of a target analyte having at least two portions comprising:
a. providing a type of nanoparticle having specific binding complements of a target analyte attached thereto, the specific binding complements being capable of recognizing at least two different portions of the target analyte;
b. forming a light scattering complex by contacting a sample believed to contain the target analyte with the nanoparticle and with a reagent that excludes volume under conditions effective to allow binding of the specific binding complement to two or more portions of the target analyte;
c. illuminating the light scattering complex under conditions effective to produce scattered light from said complex; and
d. detecting the light scattered by said complex as a measure of the presence of the target analyte.

103. The method of claim 102, wherein the reagent that excludes volume is a polymer.

104. The method of claim 102, wherein the polymer that excludes volume is a neutral or polyanionic polysaccharide.

105. The method of claim 102, wherein the neutral or polyanionic polysaccharide is a dextran sulfate polymer.

106. The method of claim 102, wherein the target analyte is a bacterial cell containing surface antigens, and the specific binding complement is an antibody or aptamer.

107. The method of claim 102, wherein the sample believed to contain the target analyte is a whole blood sample, and the contacting of the nanoparticles having specific binding complements attached thereto and detection takes place without isolation of the specific binding complement from the whole blood sample.

108. The method of claims 76 or 102, wherein the sample believed to contain the single target molecule or target analyte is a bacterial sample from a swab, culture, cellular extract, or lysed cells.

109. The method of claim 76 or 102, wherein the sample believed to contain the target analyte is a bacterial sample placed into a solution or onto a surface from a swab, culture, cellular extract, or lysed cells, and the contacting of the nanoparticles having specific binding complements attached thereto and detection takes place without isolation of the specific binding complement.

110. A method of detecting for the presence or absence of a target analyte having at least two portions comprising:
a. providing a type of nanoparticle having specific binding complements of a target analyte attached thereto, the specific binding complements being capable of recognizing at least two different portions of the target analyte;
b. forming a light scattering complex by contacting a sample believed to contain the target analyte with the nanoparticle and with a reagent that accelerates DNA renaturation under conditions effective to allow binding of the specific binding complement to two or more portions of the target analyte;
c. illuminating the light scattering complex under conditions effective to produce scattered light from said complex; and
d. detecting the light scattered by said complex as a measure of the presence of the target analyte.

111. The method of claim 110, wherein the reagent that accelerates DNA renaturation is a polymer.

112. The method of claim 110, wherein the polymer that accelerates DNA renaturation is a neutral or polyanionic polysaccharide.

113. The method of claim 111, wherein the neutral or polyanionic polysaccharide is a dextran sulfate polymer.

114. The method of claim 110, wherein the sample believed to contain the target analyte is a bacterial sample from a swab, culture, cellular extract, or lysed cells.

115. The method of claim 110, wherein the sample believed to contain the target analyte is a nucleic acid sample from lysed cells, and the contacting of the nanoparticles having specific binding complements attached thereto and detection takes place without isolation of the nucleic acid sample from the lysed cells.

116. The method of claim 102 or 110, wherein said detecting step comprises observing the wavelength and intensity of scattered light from the light scattering complex.

117. The method of claim 76, 102, or 110, further comprising providing one or more intermediate oligonucleotides, each of which comprises a first portion complementary to the target analyte, and a second portion complementary to a binding complement of a nanoparticle, wherein the intermediate oligonucleotide can bind to the target analyte and the nanoparticle binding complement.

118. The method of claim 76, 102, or 110 further comprising providing one or more intermediate probes comprising a protein that has a first portion that can bind to the target analyte, and a second portion that can bind to a binding complement of a nanoparticle, wherein the intermediate oligonucleotide can bind to the target analyte and the nanoparticle binding complement.

119. A method of detecting for the presence or absence of a single target molecule comprising:
a. providing a type of nanoparticle having specific binding complements of a single target molecule attached thereto, the specific binding complements being capable of recognizing at least two different portions of the single target molecule;
b. forming a light scattering complex by contacting a sample believed to contain the single target molecule with the nanoparticles under conditions effective to allow binding of the specific binding complements to two or more portions of the single target molecule;
c. illuminating the light scattering complex under conditions effective to produce scattered light from said complex; and
d. detecting the light scattered by said light scattering complex as a measure of the presence of the single target molecule.

120. A method of detecting for the presence or absence of a target analyte having at least two portions comprising:
a. providing at least two types of nanoparticles having specific binding complements of a target analyte attached thereto, the specific binding complements of each type of nanoparticles being capable of recognizing different portions of the target analyte;
b. forming a light scattering complex by contacting a sample believed to contain the target analyte with the nanoparticle and with a reagent that excludes volume under conditions effective to allow binding of the specific binding complement to two or more portions of the target analyte;
c. illuminating the light scattering complex under conditions effective to produce scattered light from said complex; and
d. detecting the light scattered by said complex as a measure of the presence of the target analyte.

121. A method of detecting for the presence or absence of a target analyte having at least two portions comprising:
a. providing at least two types of nanoparticles having specific binding complements of a target analyte attached thereto, the specific binding complements on each type of nanoparticles being capable of recognizing different portions of the target analyte;
b. forming a light scattering complex by contacting a sample believed to contain the target analyte with the nanoparticle and with a reagent that accelerates DNA renaturation under conditions effective to allow binding of the specific binding complement to two or more portions of the target analyte;
c. illuminating the light scattering complex under conditions effective to produce scattered light from said complex; and
d. detecting the light scattered by said complex as a measure of the presence of the target analyte.
